SEQUENCE LISTING (41 sequences)

SEQ ID NO: 1 - protein, 131 aa, Solanum chacoense
MSIPQFLGEGTPVKFVRKSESDYGDVVRVMTGVYIKFFVKTTKLCVDQTVWKVNHEGLVVTGGQVGNENDIFKIRKTDLVTPEGSKFVYKLLHCPSHLQCKNIGGNFKNGYPRLVTVDDEKDFLPFVFIKA

SEQ ID NO: 2 - protein, 217 aa, Solanum chacoense
MKSINILMFLLVSSTLSFCQSFIWDNNPIVLPTTTHDDNLVLPAVLDQDGDPVKIGKAYIIESPIIEGGSVYLDNIGGHKCPNDVLLHIPYLGLTGDGTPVKFILNDDDDLVLREMYAIHIMFSVRTSSCVNQTVWKVGDEKDFLVTGGTLGNENTFFKIMKIEMEIPDLKNVYKLLYCPPQLVCKDVSGGFDNGYPRLQVLEDDPFPYVFVKAKDA

SEQ ID NO: 3 - protein, 107 aa, Solanum chacoense
MESKFAHIIVFFLLATSFETLLARKESDGPEVIELLKEFECNGKQFWPELIGVPTKLAKEIIEKENSLINNVQILLNGSPVTMDYRCDRVRLFDNILGDVVQIPRVA

SEQ ID NO: 4 - protein, 222 aa, Solanum chacoense
MKSINILSFLLLSSTLSLVAFARSFSSENPIVLPSTCHDDDNLVLPEVYDQDGHPLRIGQRYIINNPLIGAGAVYLYNIGNLQCPNAVLQHMSIPQFLGEGTPVVFVRKSESDYGDVVRVMTAVYIKFFVKTTKLCVDQTVWKVNHEQLVVTGGEVGNENDIFKIRKTDLVTPGGSKFVYKLLHCPSHLGCKNIGGSFKNGYPRLVTVDDDKDFIPFVFIKA

SEQ ID NO: 5 - protein, 213 aa, Solanum chacoense
MKIIVVLFVSLAIFLPLASCNNNQLLVDVLDIDGKPLNASLRYHILPAYYRMRDGGVRLANLGDQSQNTCPTSVVQSKDVTDNGIAVYFTPKDRTHEHIVANSPVSIRFYLDNFNPCSNLMVWKVDHLREDVPLNGHTISTGEELVNPADHVSRWFQIEPVANNKYKLVFCPRIKCYDIGGFRQSGYNRLGLSETPIYFQFKLHPPYGKTQAY

SEQ ID NO: 6 - DNA, 396 bp, Solanum chacoense
atgtcgattccccaatttttgggagaaggcacgcccgtcaagttcgttcgtaagtcggagtcggattatggtgatgtggtgcgtgtaatgactggtgtttatatcaaattctttgttaaaacaacaaagttgtgtgttgaccaaactgtttggaaagttaatcatgaagggttggtggtaactggtggtcaggtaggaaatgaaaacgacatcttcaagattaggaaaactgacttggtgacaccagaaggttccaaatttgtatacaagttactgcattgtccctctcatcttcagtgcaaaaatatcggcggcaactttaaaaatggatatcctcgtctggtgactgtcgatgatgaaaaggactttcttccatttgtgttcatcaaggcgtag

SEQ ID NO: 7 - DNA, 654 bp, Solanum chacoense
atgaaatccattaatattttgatgttcctcttggtttcaagtaccctctccttttgtcaatctttcatttgggacaataatccaattgtcctgccgacaactactcatgatgataatcttgtactccctgccgtacttgatcaagacggcgatccagtcaagattggcaaggcctacattattgagagtcccatcatcgaaggtggatctgtatacttggataatattggaggtcataaatgcccaaacgatgtgttgctgcatattccgtatctcggtttaacgggagatggcacacccgtaaagttcattcttaatgatgatgatgatttggtactgcgtgaaatgtatgccatacatatcatgttctcggttagaacatcatcgtgtgttaaccaaactgtttggaaagttggtgatgaaaaggacttcctcgtaactggtggtaccctaggaaatgaaaataccttctttaagattatgaaaattgagatggaaataccagatttgaaaaatgtatacaagttactgtattgtccccctcaacttgtgtgtaaagatgtcagcgggggctttgataatggatatccccgtctgcaggttctcgaagacgacccttttccgtatgtcttcgtaaaggcaaaggatgcttag

SEQ ID NO: 8 - DNA, 324 bp, Solanum chacoense
atggagtcaaagtttgctcacatcattgttttctttcttcttgcaacttcctttgaaactctcttggcacgaaaagaaagtgatggaccagaagtcatagaacttctaaaggaatttgaatgcaatggaaaacaattctggccagaacttattggtgtaccaacaaagcttgctaaggaaataattgagaaggaaaattcactcataaataatgttcagatattattgaatggttctccagtcacaatggattatcgttgtgatcgagttcgtctttttgataacattttgggtgatgttgtacaaattcctagggtggcttaa

SEQ ID NO: 9 - DNA, 669 bp, Solanum chacoense
atgaagtcgattaatattttgagtttcctcttgctttcaagtaccctctctttggttgcctttgctcgatctttcagttctgagaatccaattgtcctcccctcaacttgtcatgatgatgataatcttgtactccctgaagtttatgaccaagacggccatccgctgaggattggtcagaggtacattattaacaatcctctcatcggggccggagccgtatacttgtacaatattggaaaccttcaatgcccaaacgccgtgttgcagcacatgtccattccccaatttttgggagaaggcacgcccgtcgtgttcgttcgtaagtcggagtcggattatggtgatgtggtgcgtgtaatgactgctgtttatatcaaattctttgttaaaacaacaaagttgtgtgttgaccaaactgtttggaaagttaatcatgaacagttggtggtaactggtggtgaggtaggaaatgaaaacgacatcttcaagattaggaaaactgacttggtgacaccaggaggttccaaatttgtatacaagttactgcattgtccctctcatcttgggtgcaaaaatatcggcggcagctttaaaaatggatatcctcgtctggtgactgtcgatgacgataaggactttattccatttgtgttcatcaaggcgtag

SEQ ID NO: 10 - DNA, 642 bp, Solanum chacoense
atgaagatcatagtagtactctttgtttcccttgcaatattccttccattggcaagttgcaacaacaaccaactattggtagatgtacttgatatcgatggtaaacccctcaacgcaagccttaggtatcacatacttccggcctactacaggatgagagatggaggcgtacgccttgctaatcttggggatcaatcacaaaacacttgtccaacatcggtcgtgcaatcaaaagatgttacagacaatggtatcgcagtttattttacgcctaaagatcgaacacatgagcatattgtagcaaactctccggtaagcatcagattctatcttgataattttaatccatgttctaacctaatggtgtggaaggtcgaccaccttcgtgaagacgtgccgctaaatggccacaccattagcacgggtgaagagttggtaaatcccgctgatcatgtgagtagatggttccaaattgagcctgttgcgaataacaagtataagctagtgttttgtccccgcataaaatgctatgatattggcggttttcgtcaaagtggatataatcgtttgggcctctcggagacaccaatatactttcagttcaaacttcatcctccctatggaaagacccaagcttattga

SEQ ID NO: 11 - DNA, 894 bp, Solanum chacoense
  misc_feature (2)..(2): n is a, c, g, or t
ancgattaatattttgagtttcctcttgctttcaagtaccctctctttggttgcctttgctcgatctttcagttctgagaatccaattgtcctccccacaacttgtcatgatgatgataatcttgtactccctgaagtttatgaccaaaacggccatccgctgaggattggtgagaggtacattattaacaatcctctcatcggggccggagccgtatacttgtacaatattggaaaccttcaatgcccaaacgccgtgttgcagcacatgtcgattccccaatttttgggagaaggcacgcccgtcaagttcgttcgtaagtcggagtcggattatggtgatgtggtgcgtgtaatgactggtgtttatatcaaattctttgttaaaacaacaaagttgtgtgttgaccaaactgtttggaaagttaatcatgaagggttggtggtaactggtggtcaggtaggaaatgaaaacgacatcttcaagattaggaaaactgacttggtgacaccagaaggttccaaatttgtatacaagttactgcattgtccctctcatcttcagtgcaaaaatatcggcggcaactttaaaaatggatatcctcgtctggtgactgtcgatgatgaaaaggactttcttccatttgtgttcatcaaggcgtagaatgctaattagctggctagcttgcagctttctaaataaagtgggggatatatccttctatcgctccatgtaatttaatgtatgcttatcaataaataaacaagctagcaattagcctattaccttaccttaccttcttcattcaaaaatacataaatttcttcagtttgtggtttctttttaagttagggttacttttgtttttgttgttaatcaatcgtctcgatata

SEQ ID NO: 12 - DNA, 877 bp, Solanum chacoense
aaagcaaaatacacacacaagacagtcaaattaaaaatgaaatccattaatattttgatgttcctcttggtttcaagtaccctctccttttgtcaatctttcatttgggacaataatccaattgtcctgccgacaactactcatgatgataatcttgtactccctgccgtacttgatcaagacggcgatccagtcaagattggcaaggcctacattattgagagtcccatcatcgaaggtggatctgtatacttggataatattggaggtcataaatgcccaaacgatgtgttgctgcatattccgtatctcggtttaacgggagatggcacacccgtaaagttcattcttaatgatgatgatgatttggtactgcgtgaaatgtatgccatacatatcatgttctcggttagaacatcatcgtgtgttaaccaaactgtttggaaagttggtgatgaaaaggacttcctcgtaactggtggtaccctaggaaatgaaaataccttctttaagattatgaaaattgagatggaaataccagatttgaaaaatgtatacaagttactgtattgtccccctcaacttgtgtgtaaagatgtcagcgggggctttgataatggatatccccgtctgcaggttctcgaagacgacccttttccgtatgtcttcgtaaaggcaaaggatgcttagttcatttaatttcctctatttatctttgttttctaaataaagtgctccatgtaatttcaattaattaagtaatgcatgcttatcaaaatacaaccctacctaccttacctttttgtacgtggttagctttttaatgtgtagaataagcataattaaaaattacgattggtcaacgcgcgtgttaaaa

SEQ ID NO: 13 - DNA, 540 bp, Solanum chacoense
aggcaacctctgcctataaatttatgtgatgcactcatacaaattcactcaattccttctactctttacaactaaaagaaaatggagtcaaagtttgctcacatcattgttttctttcttcttgcaacttcctttgaaactctcttggcacgaaaagaaagtgatggaccagaagtcatagaacttctaaaggaatttgaatgcaatggaaaacaattctggccagaacttattggtgtaccaacaaagcttgctaaggaaataattgagaaggaaaattcactcataaataatgttcagatattattgaatggttctccagtcacaatggattatcgttgtgatcgagttcgtctttttgataacattttgggtgatgttgtacaaattcctagggtggcttaattaatggattattattgaagtaattaagcagccacatgtcaaaaataattagggttcatgtatgttcattataatgtcttcatgtactcttactatatataatgaaataaataagtgtggcttaattaaatgtac

SEQ ID NO: 14 - DNA, 1310 bp, Solanum chacoense
taattttaccaactactctagttgaagccaaattaaataataaatatcctacaaattgaattattggccattttttatttttactcaaaatttattatttaaaatttattttggagacaaataagaaatgtctttatttaatttgatacttgagtgtaacacatattatgtactatatatttttgttgatagtaaaaaaaagaaaaggtgttagagtgacttaatattgaagcttgatattagttatttaaaatgaataaaacttatttgaggtgtctaaggcggtagtaggtattcaatttttgtttgtcaaaaatgcccttagcaggtctataaattggacatagactcatagcaagcaaaacacacaaacaaagatgaagtcgattaatattttgagtttcctcttgctttcaagtaccctctctttggttgcctttgctcgatctttcagttctgagaatccaattgtcctcccctcaacttgtcatgatgatgataatcttgtactccctgaagtttatgaccaagacggccatccgctgaggattggtcagaggtacattattaacaatcctctcatcggggccggagccgtatacttgtacaatattggaaaccttcaatgcccaaacgccgtgttgcagcacatgtccattccccaatttttgggagaaggcacgcccgtcgtgttcgttcgtaagtcggagtcggattatggtgatgtggtgcgtgtaatgactgctgtttatatcaaattctttgttaaaacaacaaagttgtgtgttgaccaaactgtttggaaagttaatcatgaacagttggtggtaactggtggtgaggtaggaaatgaaaacgacatcttcaagattaggaaaactgacttggtgacaccaggaggttccaaatttgtatacaagttactgcattgtccctctcatcttgggtgcaaaaatatcggcggcagctttaaaaatggatatcctcgtctggtgactgtcgatgacgataaggactttattccatttgtgttcatcaaggcgtagaatgctaattagctggctagcttgcagctttttaaataaagtgggggatatatccttctatcgctccatgtaaattaatgtatgcttatcaataaataaacaagctagcaattagcctattaccttaccttaccttcttcattcaaaaatacataaatttcttcagtttgtggtttctttttaagttagggttacttttgtttttgttgttaaccaatcgtctcgatatagttacttgttacacaattaatagtaatacaaaag

SEQ ID NO: 15 - DNA, 926 bp, Solanum chacoense
aaacaaaaaaaaacctaaagatgaagatcatagtagtactctttgtttcccttgcaatattccttccattggcaagttgcaacaacaaccaactattggtagatgtacttgatatcgatggtaaacccctcaacgcaagccttaggtatcacatacttccggcctactacaggatgagagatggaggcgtacgccttgctaatcttggggatcaatcacaaaacacttgtccaacatcggtcgtgcaatcaaaagatgttacagacaatggtatcgcagtttattttacgcctaaagatcgaacacatgagcatattgtagcaaactctccggtaagcatcagattctatcttgataattttaatccatgttctaacctaatggtgtggaaggtcgaccaccttcgtgaagacgtgccgctaaatggccacaccattagcacgggtgaagagttggtaaatcccgctgatcatgtgagtagatggttccaaattgagcctgttgcgaataacaagtataagctagtgttttgtccccgcataaaatgctatgatattggcggttttcgtcaaagtggatataatcgtttgggcctctcggagacaccaatatactttcagttcaaacttcatcctccctatggaaagacccaagcttattgatcatcatcatatattacatgtatgcctcgatttgctctcgaatgaaatcatgactatgtaatatgtaataataaggaccttagtccaaataataataatgtaatgcatggcctagataagtgccaatgcacactactttatctttgatgttgctcttctttttaatttccgcaaagtaaaagtaaattaaagaaaatattaacccataagcatctcttggactcaaattaatctcatgaaccatcagattaaaaatcaattggc

SEQ ID NO: 16 - DNA, 20 bp, Pectobacterium brasiliense
agcgacgaactgggtgaatt

SEQ ID NO: 17 - DNA, 20 bp, Pectobacterium brasiliense
ccctctgagcgccactttta

SEQ ID NO: 18 - DNA, 20 bp, Pectobacterium brasiliense
ggacgcagcttcttctcaca

SEQ ID NO: 19 - DNA, 20 bp, Pectobacterium brasiliense
ggctgatctttcacgtcggt

SEQ ID NO: 20 - DNA, 20 bp, Pectobacterium brasiliense
gctgatggaacgtgtggagt

SEQ ID NO: 21 - DNA, 20 bp, Pectobacterium brasiliense
gccaaattgcagaatcgcca

SEQ ID NO: 22 - DNA, 20 bp, Pectobacterium brasiliense
agtctcaactcgccgatctg

SEQ ID NO: 23 - DNA, 20 bp, Pectobacterium brasiliense
agaccttccaacttgacgcc

SEQ ID NO: 24 - DNA, 20 bp, Pectobacterium brasiliense
ctggatgtggcgaccgataa

SEQ ID NO: 25 - DNA, 20 bp, Pectobacterium brasiliense
acaccttgccccagattcag

SEQ ID NO: 26 - DNA, 20 bp, Pectobacterium brasiliense
caaccaaagtcaccagcacg

SEQ ID NO: 27 - DNA, 20 bp, Pectobacterium brasiliense
tgcactggttttggctgttg

SEQ ID NO: 28 - DNA, 20 bp, Pectobacterium brasiliense
tctgcggtgggtaacttgac

SEQ ID NO: 29 - DNA, 20 bp, Pectobacterium brasiliense
tcaccgctaaaccttgggac

SEQ ID NO: 30 - DNA, 20 bp, Pectobacterium brasiliense
gcacgtttagcaatcaggcc

SEQ ID NO: 31 - DNA, 20 bp, Pectobacterium brasiliense
gtaaggctgggtgaacgaca

SEQ ID NO: 32 - DNA, 25 bp, Artificial Sequence
  Primer
cgccatatgatgtcgattccccaat

SEQ ID NO: 33 - DNA, 25 bp, Artificial Sequence
  Primer
ggaggatccctacgccttgatgaac

SEQ ID NO: 34 - DNA, 33 bp, Artificial Sequence
  Primer
cgccatatgatgaaatccattaatattttgatg

SEQ ID NO: 35 - DNA, 26 bp, Artificial Sequence
  Primer
ggaggatccctaagcatcctttgcct

SEQ ID NO: 36 - DNA, 26 bp, Artificial Sequence
  Primer
cgccatatgatggagtcaaagtttgc

SEQ ID NO: 37 - DNA, 25 bp, Artificial Sequence
  Primer
ggaggatccttaagccaccctagga

SEQ ID NO: 38 - DNA, 32 bp, Artificial Sequence
  Primer
cgccatatgatgaagtcgattaatattttgag

SEQ ID NO: 39 - DNA, 26 bp, Artificial Sequence
  Primer
ggaggatccctacgccttgatgaaca

SEQ ID NO: 40 - DNA, 30 bp, Artificial Sequence
  Primer
cgccatatgatgaagatcatagtagtactc

SEQ ID NO: 41 - DNA, 27 bp, Artificial Sequence
  Primer
ggaggatcctcaataagcttgggtctt

(19) United States
(12) Patent Application Publication
(10) Pub. No.: US 2025/0290087 A1
(43) Pub. Date: Sep. 18, 2025

(54) PROTEASE INHIBITORS AND THEIR USE TO PROVIDE DISEASE RESISTANCE IN PLANTS AND AS ANTIMICROBIALS

(71) Applicant: COLORADO STATE UNIVERSITY RESEARCH FOUNDATION, Fort Collins, CO (US)

(72) Inventors: Adam L. Heuberger, Fort Collins, CO (US); Amy O. Charkowski, Fort Collins, CO (US); Janak R. Joshi, Fort Collins, CO (US)

(21) Appl. No.: 18/256,396

(22) PCT Filed: Dec. 10, 2021
(86) PCT No.: PCT/US2021/062804

§ 371 Date: Jun. 7, 2023

Related U.S. Application Data
(60) Provisional application No. 63/123,611, filed on Dec. 10, 2020.
(60) Provisional application No. 63/261,771, filed on Sep. 28, 2021.

(51) Int. Cl. C12N 15/82; A01N 37/46; A01P 1/00; C07K 14/415

(52) U.S. Cl. CPC C12N 15/8281; A01N 37/46; A01P 1/00; C07K 14/415

(57) ABSTRACT

Compositions and methods for enhancing the resistance of plants to a disease caused by a bacterial pathogen are provided. The compositions comprise nucleic acid molecules encoding protease inhibitor gene products and variants thereof and plants, seeds, and plant cells comprising such nucleic acid molecules. The methods for enhancing the resistance of a plant to a disease caused by a bacterial pathogen comprise introducing a nucleic acid molecule encoding a protease inhibitor gene product into a plant cell. Additionally provided are antimicrobial compositions containing one or more protease inhibitor proteins for controlling antimicrobial growth on plants or plant parts, and methods for controlling or preventing the growth of microbial pathogens, and in particular bacterial pathogens, on plants and plant parts.

### CROSS REFERENCE TO RELATED APPLICATIONS

This application claims priority to provisional applications U.S. Ser. No. 63/123,611 filed Dec. 10, 2020 and U.S. Ser. No. 63/261,771 filed Sep. 28, 2021, which are incorporated herein by reference in their entireties.

### GRANT REFERENCE

This invention was made with government support under grant 2017-51181-26827 awarded by USDA NIFA. The US government has certain rights in the invention.

### SEQUENCE LISTING

The instant application contains a Sequence Listing which has been submitted in ASCII format via Electronic Submission and is hereby incorporated by reference in its entirety. Said ASCII copy, created on Nov. 27, 2021, is named P13706WO00_ST25.txt and is 23,288 bytes in size.

### TECHNICAL FIELD

The present disclosure relates to the field of biotechnology. More specifically, the present disclosure relates to enhancing the resistance of plants to disease, particularly disease caused by bacterial pathogens.

### BACKGROUND

Bacterial pathogens of plants are major threats to global food security. Compared to fungi and pests, chemical control is difficult and management relies primarily on plant resistance, sanitation, and exclusion. The plant immune system is innate and acts to induce bacterial cell death via toxins, by programmed plant cell death and isolation of bacteria, or by reallocating nutrients away from infection sites to slow pathogen multiplication. In some plant-bacterial pathogenesis systems, the plant recognizes the presence of pathogens via cell surface receptors or cytoplasmic resistance proteins. However, there is high selection pressure for bacteria to evade detection, so recognition-based immunity can be short-lived. Alternatively, some plants synthesize bactericidal compounds even in the absence of disease, such as metabolites or small peptides that are stored in the plant cell vacuole and are only released when cell membranes are disrupted. However, an immune response reliant on toxins also leads to high selection pressure, and bacteria often overcome this method of resistance by acquiring toxin transporter or detoxification systems. Identifying durable sources of disease resistance is therefore an important area of research in plant biology.

As the fourth largest crop consumed worldwide, it is important to develop durable disease resistance in potato (Solanum tuberosum L.). Modern cultivated potato is highly susceptible to the bacterial necrotrophic pathogen Pectobacterium, which causes soft rot (a tuber infection), aerial stem rot (invasion of wounded stems), and blackleg disease (early wilting from bacterial transfer from tubers to stems). The symptoms of these diseases (rotting, wilting, blackening) are caused primarily by plant cell wall degrading enzymes (PCWDEs) secreted by these pathogens, such as pectate lyase and protease. Expression of PCWDE genes is tightly regulated by acyl-homoserine lactone (AHL)-based quorum-sensing and detection of plant organic acids and plant cell wall fragments. In addition to potato, Pectobacterium infects crops in up to 50% of angiosperm plant orders. There are no curative measures for diseases caused by this pathogen, and it is common in irrigation water and present worldwide. Therefore, Pectobacterium is considered among the most important plant pathogens.

### SUMMARY

The present disclosure provides nucleic acid molecules for protease inhibitor genes that are capable of conferring to a plant, particularly a potato plant, resistance to at least one bacterial pathogen that is known to cause a plant disease in the plant. In one embodiment, the present disclosure provides nucleic acid molecules comprising a protease inhibitor gene, which is referred to herein as g18987, and its variants including, for example, alleles of g18987, homologs of g18987, and other naturally and non-naturally occurring variants of g18987. In another embodiment, the present disclosure provides nucleic acid molecules comprising a protease inhibitor gene, which is referred to herein as g28531, and its variants including, for example, alleles of g28531, homologs of g28531, and other naturally and non-naturally occurring variants of g28531. In yet another embodiment, the present disclosure provides nucleic acid molecules comprising a protease inhibitor gene, which is referred to herein as g39249, and its variants including, for example, alleles of g39249, and homologs of g39249, and other naturally and non-naturally occurring variants of g39249. In a further embodiment, the present disclosure provides nucleic acid molecules comprising a protease inhibitor gene, which is referred to herein as g40384, and its variants including, for example, alleles of g40384, homologs of g40384, and other naturally and non-naturally occurring variants of g40384. In a yet further embodiment, the present disclosure provides nucleic acid molecules comprising a protease inhibitor gene, which is referred to herein as g6571, and its variants including, for example, alleles of g6571, homologs of g6571, and other naturally and non-naturally occurring variants of g6571.

The present disclosure additionally provides plants, plant cells, and seeds comprising in their genomes one or more heterologous polynucleotides of the disclosure. The heterologous polynucleotides comprise a nucleotide sequence encoding a protease inhibitor protein of the present disclosure. Such protease inhibitor proteins are encoded by the protease inhibitor genes of the present disclosure, particularly g18987, g28531, g39249, g40384, and g6571, and alleles, homologs, and other naturally and non-naturally occurring variants of such protease inhibitor genes. In certain embodiment, the plants and seeds are transgenic potato plants and seeds that have been transformed with one or more heterologous polynucleotides of the disclosure. Preferably, such potato plants comprise enhanced resistance to at least one bacterial pathogen that is known to cause a plant disease in a potato plant, when compared to the resistance of a control plant that does not comprise the heterologous polynucleotide.

The present disclosure provides methods for enhancing the resistance of a plant, particularly a potato plant, to a plant disease caused by at least one bacterial pathogen. Such methods comprise introducing into at least one plant cell a heterologous polynucleotide comprising a nucleotide sequence of a protease inhibitor gene of the present disclosure. Preferably, the heterologous polynucleotide or part thereof is stably incorporated into the genome of the plant cell. The methods can optionally further comprise regenerating the plant cell into a plant that comprises in its genome the heterologous polynucleotide. Preferably, such a plant comprises enhanced resistance to a plant disease caused by at least one bacterial pathogen, relative to a control plant not comprising the heterologous polynucleotide.

The present disclosure additionally provides methods for identifying a plant, particularly a potato plant, that displays newly conferred or enhanced resistance to a plant disease caused by at least one bacterial pathogen. The methods comprise detecting in the plant the presence of g18987, g28531, g39249, g40384, and/or g6571, and/or alleles, homologs, and other naturally and non-naturally occurring variants of such protease inhibitor genes.

Methods of using the plants of the present disclosure in agricultural crop production to limit plant disease caused by at least one bacterial pathogen are also provided. The methods comprise planting a plant (e.g. a seedling), a tuber, or a seed of the present disclosure, wherein the plant, tuber, or seed comprises at least one protease inhibitor gene nucleotide sequence of the present disclosure. The methods further comprise growing a plant under conditions favorable for the growth and development of the plant, and optionally harvesting at least one fruit, tuber, leaf, or seed from the plant.

Additionally provided are plants, plant parts, seeds, plant cells, other host cells, expression cassettes, and vectors comprising one or more of the nucleic acid molecules of the present disclosure.

The present disclosure provides an antimicrobial composition, the composition comprising at least one protease inhibitor protein of the present disclosure as an active agent. The antimicrobial composition may contain two, three, four, five, or more of the protease inhibitor proteins of the present disclosure. The antimicrobial composition may be an antibacterial composition for controlling the growth of one or more bacterial pathogens on plants or plant parts, including harvested plant parts. The antimicrobial composition may be capable of treating or preventing a bacterial soft rot.

The present disclosure additional provides a method of preventing or controlling microbial growth on plants or plant parts, the method comprising contacting the antimicrobial composition of the present disclosure to the plants or plant parts.

While multiple embodiments are disclosed, still other embodiments of the inventions will become apparent to those skilled in the art from the following detailed description, which shows and describes illustrative embodiments of the invention. Accordingly, the figures and detailed description are to be regarded as illustrative in nature and not restrictive.

### BRIEF DESCRIPTION OF THE FIGURES

The following drawings form part of the specification and are included to further demonstrate certain embodiments or various aspects of the invention. In some instances, embodiments of the invention can be best understood by referring to the accompanying figures in combination with the detailed description presented herein. The description and accompanying figures may highlight a certain specific example, or a certain aspect of the invention. However, one skilled in the art will understand that portions of the example or aspect may be used in combination with other examples or aspects of the invention.

FIG. 1A-D shows effects of potato protein extracts on virulence traits of P. brasiliense Pb1692. FIG. 1A shows Log₁₀ CFU counts of Pb1692 in protein extracts of DM1, M6, or buffer (negative control) after 15 h of incubation with 400 μg·ml⁻¹ of protein extract had no effect on multiplication. FIG. 1B shows Pb1692 pectate lyase and protease activity when cultured in nutrient broth containing 400 μg·ml⁻¹ protein extract or extraction buffer. FIG. 1C shows dose-dependent effect of M6 protein extract on exo-enzyme activity. FIG. 1D shows expression level of Pb1692 PCWDE genes when cultured in nutrient broth containing protein extract or buffer. Data is presented as mean±SEM, which is a combination of two independent biological experiments, with n=4 (for a, b, and d) and n=3 (for c) replicates per experiment. Asterisk indicates difference between treatment and buffer control (ANOVA Dunnett's post hoc, p<0.05); ns=not significant; in c, letters indicate differences determined by ANOVA Tukey post hoc p<0.05.

FIG. 2A-B shows unique effects of M6 protein extract on the P. brasiliense Pb1692 proteome. Pb1692 protein levels and gene expression was measured following incubation with M6 protein extracts and compared to DM1 as a susceptible control. FIG. 2A is a volcano plot showing differential abundance (log₂ M6/DM1, x-axis) and significance (−log₁₀ p-value, y-axis) of Pb1692. Colored circles indicate significant proteins affected by M6 protein extract for intra (blue) and extracellular (orange) protein data (log₂>0.6 or <−0.6; (−log₁₀ p-value >1.3). FIG. 2B expression level of Pb1692 virulence (vir.) genes in response to treatment with M6 compared to DM1. The bacterial cells (10⁶ CFU ml⁻¹) were grown in protein extracts (400 μg·ml⁻¹) for 15 h at 28° C. and transcript level of srfB (virulence factor); pemA (pectinesterase A); nfuA (Fe/S biogenesis protein); metP (metalloprotease); fruB (phosphocarrier protein); fliD (flagellar filament capping protein); cheA (chemotaxis protein); artI (arginine ABC transporter) were determined by qRT-PCR. The transcript levels were normalized to the central metabolism gene recA and transformed relative to DM1 [log₂ (relative expression)]. Data shown are the combination of two independent experiments with n=3 replicates in each experiment and presented as mean±SEM.

FIG. 3A-D shows proteome and expressed protease inhibitor (PI) genome variation between susceptible (DM1) and resistant (M6) potato. FIG. 3A is a volcano plots showing differential abundance (log₂ M6/DM1, x-axis) and significance (−log₁₀ p-value, y-axis) of tuber proteins/peptides. Colored circles indicate differentially expressed protein between DM1 and M6 (fold change greater or less than 1.5; Student t-test p<0.05). Pink triangles denote PIs. FIG. 3B shows phylogenetic analysis of PIs expressed in DM1 and M6 tuber, and representative two tomato PIs (names italicized) of respective families. The tree is based on protein sequence alignment and colors indicate PI family. Bootstrap values (500 replicates) are near each node. FIG. 3C shows the location of expressed PIs is shown across potato chromosomes with color denoting PI family. Asterisk indicates cloned genes. FIG. 3D shows domain analysis of protease inhibitor genes in DM1 and M6. Protein sequences were assigned to domains based on PI type/domain in Simple Modular Architecture Research Tool-SMART. Asterisk indicates cloned genes.

FIG. 4A-F shows protease and motility inhibition and cell morphology effects of M6 potato protein on P. brasiliense Pb1692. Pb1692 cultures were incubated with 400 μg·ml⁻¹ DM1 or M6 protein and compared to cultures with protein extraction buffer as a negative control and or buffer with a protease inhibitor cocktail (cPI). FIG. 4A shows trypsin inhibition activity. FIG. 4B shows exo-protease inhibition. FIG. 4C shows motility inhibition. Y-axis area measurements indicate spread of activity halo on agar plates. Data are presented as mean±SEM and are a combination of two independent experiments, with n=8 for (a and b) and n=5 for I. Asterisk indicates differences between treatment and buffer control (ANOVA Dunnett's post hoc, p<0.05). FIG. 4D shows example observations of Pb1692 under compound microscope (1000×). Bacterial cells were exposed to controls or potato protein extracts, fixed on glass slide, and stained with crystal violet. Representative filamentous cells (>5 μm) are marked with yellow arrows. Microscopy experiments were repeated 3 times with similar results.

FIG. 5 shows representative images of protease activity measured in milk-agar plates. Pb1692 supernatant consists of exogenous proteases (‘exo-proteases’) that can degrade milk protein (casein), resulting in appearance of haloes. A cocktail of authentic protease inhibitors (cPI) inhibited protease activity and was used as a positive control. Similar effects of exo-protease inhibition were observed from M6 (resistant potato) protein extract, and inhibition was maintained even after heating (70° C., 20 min), but not with DM1 protein extracts.

FIG. 6 shows representative images of Pectobacterium swimming motility. Overnight grown Pb1692 cells were centrifuged, and pellet were resuspended in protein extracts from S. chacoense M6 (resistant potato) and S. tuberosum DM1 (susceptible potato). A cocktail of authentic protease inhibitors (cPI) was used as a positive control. Pb1692 motility was reduced with the cPI and M6 treatments, and inhibition was maintained even after heating (70° C., 20 min), but not with DM1 protein extracts.

FIG. 7A-F shows effect of cloned and purified M6 protease inhibitors on P. brasiliense Pb1692 virulence factors and disease. Protease inhibitors were cloned from S. chacoense M6, purified, and tested for effects on trypsin activity, and Pb1692 bacterial exo-protease activity and motility. FIG. 7A shows trypsin activity. FIG. 7B shows Pb1692 bacterial exo-protease activity. FIG. 7C shows motility. Data is presented as mean±SEM, which is a combination of two independent biological replicates, with n=8 for (a and b) and n=5 for I. Asterisk indicates significant differences between treatment and empty vector control (ANOVA Dunnett's post hoc p<0.05). FIG. 7D shows observation of Pb1692 cells under compound microscope (1000×). Bacterial cells were exposed to empty vector protein extract or cloned and purified M6 PIs, fixed onto a glass slide, and stained with crystal violet. Representative filamentous cells (>5 μm) are marked with yellow arrows. Experiments were repeated 3 times and each replicate demonstrated similar results. FIG. 7E shows representative images of disease symptoms on potato tubers co-inoculated with Pb1692 and each of the purified M6 PIs. FIG. 7F shows quantitation of tuber disease severity caused by Pb1692, measured as amount of decayed tissue collected from the tuber.

FIG. 8 shows representative images of effects on cloned and purified M6 PIs on Pectobacterium swimming motility. Overnight grown Pb1692 cells were centrifuged, and pellet were resuspended in protease inhibitors (PIs). Two PIs (g28531 and g6571) significantly reduced Pb1692 motility, however others (g18987, g33601, g40384) did not have effect.

FIG. 9 is a graph of the effects on swimming motility of D. solani, E. coli RP437, and P. fluorescens.

### DETAILED DESCRIPTION

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

The term “about,” as used herein, refers to variation in the numerical quantity that can occur, for example, through typical measuring techniques and equipment, with respect to any quantifiable variable, including, but not limited to, mass, volume, time, and temperature. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

The present disclosure relates to the identification and isolation of protease inhibitor genes, particularly protease inhibitor genes that contribute to bacterial disease resistance, including necrotrophic bacterial pathogens such as Pectobacterium. As disclosed hereinbelow, protease inhibitor genes were identified in Solanum chacoense line M6, a resistant wild relative of potato.

Protease inhibitors (PIs) are a part of the innate defense strategy by plants. They are grouped into six families based on structure, sequence similarity and the type of protease they inhibit: aspartic, cysteine, metalloproteases, serine, threonine, and trypsin inhibitors. These proteins compete with protease substrates and bind in reversible or irreversible manners, and act against fungal membranes and multiplication of viruses, and are thought to affect bacterial outer-membrane structure and extracellular pathogenesis proteins. The presently disclosed S. chacoense protease inhibitors contribute to bacterial disease resistance by inhibiting exo-proteases, motility, and tuber maceration, and by modulating cell morphology and metabolism.

The present disclosure provides nucleic acid molecules comprising the nucleotide sequences of protease inhibitor genes, particularly the nucleotide sequences of g18987, g28531, g39249, g40384, and g6571 and alleles, homologs, orthologs, and other naturally occurring variants of such protease inhibitor genes and synthetic or artificial (i.e. non-naturally occurring) variants thereof. As used herein, such nucleic acid molecules are referred to herein as “protease inhibitor nucleic acid molecules” or “protease inhibitor genes”, unless stated otherwise or apparent from the context of use. Likewise, the nucleotide sequences of g18987, g28531, g39249, g40384, and g6571 and alleles, homologs, orthologs, and other naturally occurring variants of such protease inhibitor genes and synthetic or artificial (i.e. non-naturally occurring) variants thereof are referred to herein as “protease inhibitor nucleotide sequences” unless stated otherwise or apparent from the context of use.

TABLE 1
Sequences of Solanum chacoense protease inhibitors.

| Protease | | | |
|---|---|---|---|
| inhibitor | Protein sequence | Coding sequence | cDNA sequence |
| g18987 | SEQ ID NO: 1 | SEQ ID NO: 6 | SEQ ID NO: 11 |
| g28531 | SEQ ID NO: 2 | SEQ ID NO: 7 | SEQ ID NO: 12 |
| g39249 | SEQ ID NO: 3 | SEQ ID NO: 8 | SEQ ID NO: 13 |
| g40384 | SEQ ID NO: 4 | SEQ ID NO: 9 | SEQ ID NO: 14 |
| g6571 | SEQ ID NO: 5 | SEQ ID NO: 10 | SEQ ID NO: 15 |

Protease inhibitor nucleotide sequences of the disclosure include, but not limited to, the nucleotide sequences of wild-type g18987, g28531, g39249, g40384, and g6571 genes comprising a native promoter and the 3′ adjacent region comprising the coding region, cDNA sequences, and nucleotide sequences comprising only the coding region. Examples of such protease inhibitor nucleotide sequences include the nucleotide sequences set forth in SEQ ID NOs: 6-15 and variants thereof. In embodiments in which the native protease inhibitor gene promoter is not used to drive the expression of the nucleotide sequence encoding the protease inhibitor protein, a heterologous promoter can be operably linked a nucleotide sequence encoding a protease inhibitor protein of the disclosure to drive the expression of nucleotide sequence encoding a protease inhibitor protein in a plant.

Preferably, the protease inhibitor proteins encoded by the protease inhibitor nucleotide sequences of the disclosure are functional protease inhibitor proteins, or part(s), or domain(s) thereof, which are capable of conferring on a plant, particularly a potato plant, comprising the protease inhibitor protein enhanced resistance to a plant disease caused by at least one bacterial pathogen. In certain embodiments, the protease inhibitor proteins of the present disclosure are capable of conferring on a plant broad-spectrum resistance to at least one bacterial pathogen, but preferably multiple bacterial pathogens, and include, for example, g18987 (SEQ ID NO: 6) and the protease inhibitor protein encoded by g18987 (SEQ ID NO: 1). Such protease inhibitor proteins of the present disclosure include, but are not limited to, the protease inhibitor proteins comprising the amino acid sequences set forth in SEQ ID NOs: 1-5 and/or are encoded by the protease inhibitor nucleotide sequences set forth in SEQ ID NOs: 6-15.

Likewise, preferred protease inhibitor genes and protease inhibitor nucleic acid molecules of the present disclosure are capable of conferring on a plant, particularly a potato plant, comprising the protease inhibitor gene, the protease inhibitor nucleic acid molecule, or protease inhibitor allele, enhanced resistance to a plant disease caused by at least one bacterial pathogen. In certain preferred embodiments, the protease inhibitor genes and protease inhibitor nucleic acid molecules of the present disclosure are capable of conferring on a plant resistance to at least one bacterial pathogen, but preferably multiple bacterial pathogens. Such protease inhibitor genes and protease inhibitor nucleic acid molecules include, but are not limited to, protease inhibitor genes and protease inhibitor nucleic acid molecules comprising a nucleotide sequence selected from: a nucleotide sequences set forth in SEQ ID NOs: 6-15; and a nucleotide sequence encoding an amino acid sequence set forth in SEQ ID NOs: 1-5.

The present disclosure further provides plants comprising a heterologous polynucleotide which comprises a protease inhibitor gene nucleotide sequence of the present disclosure. Preferably, such a protease inhibitor gene nucleotide sequence encodes a full-length protease inhibitor protein of the present disclosure, or at least a functional part(s) or domain(s) thereof. In some embodiments, such a heterologous polynucleotide of the present disclosure is stably incorporated into the genome of the plant, and in other embodiments, the plant is transformed by a transient transformation method and the heterologous polynucleotide is not stably incorporated into the genome of the plant.

In other embodiments, a plant comprising a heterologous polynucleotide which comprises a protease inhibitor gene nucleotide sequence of the present disclosure is produced using a method of the present disclosure that involves genome editing to modify the nucleotide sequence of a native or non-native gene in the genome of the plant. The native or non-native gene comprises a nucleotide sequence that is different from (i.e. not identical to) a protease inhibitor gene nucleotide sequence of the present disclosure, and after modification by methods disclosed in further detail hereinbelow, the modified native or non-native gene comprises a protease inhibitor gene nucleotide sequence of the present disclosure. Generally, such methods comprise the use of a plant comprising in its genome a native or non-native gene wherein the native or non-native gene comprises a nucleotide sequence that is homologous to a protease inhibitor gene nucleotide sequence of the present disclosure and further comprises introducing into the plant a nucleic acid molecule comprising at least part of a protease inhibitor gene nucleotide sequence of the present disclosure. Preferably, a nucleotide sequence of native or non-native gene comprises about 70%, 75% 80%, 85%, 90%, 95%, 96%, 97%, 98%, 99% or greater nucleotide sequence identity to at least one protease inhibitor gene nucleotide sequence of the present disclosure. Such a native or non-native gene can be, for example a protease inhibitor gene, or a non-functional homolog of such a protease inhibitor gene that is not, or is not known to be, capable of conferring to a plant, resistance to a plant disease. It is recognized that a plant produced by genome engineering as disclosed herein is a stably transformed plant when the native or non-native gene that is modified is stably incorporated in the genome of the plant.

Methods for both the stable and transient transformation of plants and genome editing are disclosed elsewhere herein or otherwise known in the art. In some embodiments, the plants are stably transformed potato plants comprising a heterologous polynucleotide of the present disclosure stably incorporated into its genome and further comprising enhanced resistance to disease caused by at least one bacterial pathogen. In a more preferred embodiment, the plants are stably transformed potato plants comprising a heterologous polynucleotide of the present disclosure stably incorporated into its genome and further comprising enhanced resistance to disease caused by at least two, three, four, five, six or more bacterial pathogens.

In certain embodiments, a plant of the disclosure comprises a heterologous polynucleotide which comprises a nucleotide sequence encoding a protease inhibitor protein of the present invention and a heterologous promoter that is operably linked for expression of the nucleotide sequence encoding an R protein. The choice of heterologous promoter can depend on a number of factors such as, for example, the desired timing, localization, and pattern of expression as well as responsiveness to particular biotic or abiotic stimulus. Promoters of interest include, but are not limited to, pathogen-inducible, constitutive, tissue-preferred, wound-inducible, and chemical-regulated promoters.

In certain embodiments, a plant of the disclosure, particularly a potato plant, can comprise one, two, three, four, five, six, or more nucleotide sequences encoding a protease inhibitor protein. Typically, but not necessarily, the two or more protease inhibitor proteins will be different from each other. For the present disclosure, a protease inhibitor protein is different from another protease inhibitor protein when the two protease inhibitor proteins have non-identical amino acid sequences. In certain embodiments, each of the different protease inhibitor proteins for resistance to a plant disease caused by a bacterial pathogen has one or more differences in characteristics such as, for example, differences in inhibiting exo-proteases, inhibiting motility, inhibiting tuber maceration, modulating cell morphology, or modulating metabolism. It is recognized that by combining two, three, four, five, six, or more nucleotide sequences with each nucleotide sequence encoding a different protease inhibitor protein, a plant can be produced that comprises durable broad-spectrum resistance.

A plant of the disclosure comprising multiple protease inhibitor genes can be produced, for example, by transforming a plant that already comprises one or more other protease inhibitor gene nucleotide sequences with a heterologous polynucleotide comprising at least one protease inhibitor nucleotide sequence of the present disclosure including, for example, one or more of a g18987 nucleotide sequence, a g28531 nucleotide sequence, a g39249 nucleotide sequence, a g40384 nucleotide sequence, and a g6571 nucleotide sequence. Such a plant that already comprises one or more other protease inhibitor gene nucleotide sequences can comprise protease inhibitor genes that are native to the genome or the plant, that were introduced into the plant via sexual reproduction, or that were introduced by transforming the plant or a progenitor thereof with a protease inhibitor gene nucleotide sequence. Alternatively, the one or more other protease inhibitor gene nucleotide sequences can be introduced into a plant of the disclosure, which already comprises a heterologous polynucleotide of the disclosure, by, for example, transformation or sexual reproduction.

In other embodiments, two or more different protease inhibitor gene sequences can be introduced into a plant by stably transforming the plant with a heterologous polynucleotide or vector comprising two or more protease inhibitor gene nucleotide sequences. It is recognized that such an approach can be preferred for plant breeding as it is expected that the two or more protease inhibitor gene nucleotide sequences will be tightly linked and thus, segregate a single locus. Alternatively, a heterologous polynucleotide of the present invention can be incorporated into the genome of a plant in the immediate vicinity of another protease inhibitor gene nucleotide sequence using homologous recombination-based genome modification methods that are described elsewhere herein or otherwise known in the art.

The present disclosure further provides methods for enhancing the resistance of a plant to a plant disease caused by at least one bacterial pathogen. The methods comprise modifying at least one plant cell to comprise a heterologous polynucleotide, and optionally regenerating a plant from the modified plant comprising the heterologous polynucleotide. In a first aspect, the methods for enhancing the resistance of a plant to a plant disease caused by at least one bacterial pathogen comprise introducing a heterologous polynucleotide of the invention into at least one plant cell, particular a plant cell from a potato plant. In certain embodiments, the heterologous polynucleotide is stably incorporated into the genome of the plant cell.

In a second aspect, the methods for enhancing the resistance of a plant to a plant disease caused by at least one bacterial pathogen involve the use of a genome-editing method to modify the nucleotide sequences of a native or non-native gene in the genome of the plant cell to comprise a heterologous polynucleotide of the present invention. The methods comprise introducing a nucleic acid molecule into the plant cell, wherein the nucleic acid molecule comprises a nucleotide sequence comprising at least a part of the protease inhibitor nucleotide sequence of the present disclosure and wherein at least a part of the nucleotide sequence of the native or non-native gene is replaced with at least a part of the nucleotide sequence of the nucleic acid molecule. Thus, the methods of the disclosure involve gene replacement to produce a heterologous polynucleotide of the present disclosure in the genome of a plant cell.

If desired, the methods of the first and/or second aspect can further comprise regenerating the plant cell into a plant comprising in its genome the heterologous polynucleotide. Preferably, such a regenerated plant comprises enhanced resistance to a plant disease caused by at least one bacterial pathogen relative to the resistance of a control plant to the plant disease.

The methods of the present disclosure for enhancing the resistance of a plant to a plant disease caused by at least one bacterial pathogen can further comprise producing a plant comprising two, three, four, five, six, or more nucleotide sequences encoding a protease inhibitor protein, preferably each nucleotide sequence encoding a different protease inhibitor protein. Such a plant comprising multiple protease inhibitor gene nucleotide sequences comprises one or more additional protease inhibitor gene nucleotide sequences of the present disclosure and/or any other nucleotide sequence encoding a protease inhibitor protein known in the art. It is recognized that the methods of the first and/or second aspect can be used to produce such a plant comprising multiple nucleotide sequences encoding a protease inhibitor protein. Moreover, it is recognized that a heterologous polynucleotide of the present disclosure can comprise, for example, one or more protease inhibitor nucleotide sequences of the present protease inhibitor or at least one protease inhibitor nucleotide sequences of the present disclosure and one or more nucleotide sequences encoding a protease inhibitor protein that is known in the art.

The plants disclosed herein find use in methods for limiting plant disease caused by at least one bacterial pathogen in agricultural crop production, particularly in regions where such a plant disease is prevalent and is known to negatively impact, or at least has the potential to negatively impact, agricultural yield. The methods of the disclosure comprise planting a plant (e.g. a seedling), tuber, or seed of the present disclosure, wherein the plant, tuber, or seed comprises at least one protease inhibitor gene nucleotide sequence of the present disclosure. The methods further comprise growing the plant that is derived from the seedling, tuber, or seed under conditions favorable for the growth and development of the plant, and optionally harvesting at least one fruit, tuber, leaf, or seed from the plant.

The present disclosure additionally provides methods for identifying a plant that displays newly conferred or enhanced resistance to a plant disease caused by at least one bacterial pathogen. The methods find use in breeding plants for resistance to plant diseases caused by bacterial pathogen such as, for example, bacterial soft rot. Such resistant plants find use in the agricultural production of fruits, tubers, leaves, and/or seeds for human or livestock consumption or other use. The methods comprise detecting in a plant, or in at least one part or cell thereof, the presence of a protease inhibitor nucleotide sequence of the present disclosure. In some embodiments, detecting the presence of the protease inhibitor nucleotide sequence comprises detecting the entire protease inhibitor nucleotide sequence in genomic DNA isolated from a plant. In certain embodiments, however, detecting the presence of a protease inhibitor nucleotide sequence comprises detecting the presence of at least one marker within the protease inhibitor nucleotide sequence. In other embodiments, detecting the presence of a protease inhibitor nucleotide sequence comprises detecting the presence of the protease inhibitor protein encoded by the protease inhibitor nucleotide sequence using, for example, immunological detection methods involving antibodies specific to the protease inhibitor protein.

In the methods for identifying a plant that displays newly conferred or enhanced resistance to a plant disease caused by at least one bacterial pathogen, detecting the presence of the protease inhibitor nucleotide sequence in the plant can involve one or more of the following molecular biology techniques that are disclosed elsewhere herein or otherwise known in the art including, but not limited to, isolating genomic DNA and/or RNA from the plant, amplifying nucleic acid molecules comprising the protease inhibitor nucleotide sequence and/or marker therein by PCR amplification, sequencing nucleic acid molecules comprising the protease inhibitor nucleotide sequence and/or marker, identifying the protease inhibitor nucleotide sequence, the marker, or a transcript of the protease inhibitor nucleotide sequence by nucleic acid hybridization, and conducting an immunological assay for the detection of the protease inhibitor protein encoded by the protease inhibitor nucleotide sequence. It is recognized that oligonucleotide probes and PCR primers can be designed to identity the protease inhibitor nucleotide sequences of the present disclosure and that such probes and PCR primers can be utilized in methods disclosed elsewhere herein or otherwise known in the art to rapidly identify in a population of plants one or more plants comprising the presence of a protease inhibitor nucleotide sequence of the present disclosure.

Depending on the desired outcome, the heterologous polynucleotides of the disclosure can be stably incorporated into the genome of the plant cell or not stably incorporated into genome of the plant cell. If, for example, the desired outcome is to produce a stably transformed plant with enhanced resistance to a plant disease caused by at least one bacterial pathogen, then the heterologous polynucleotide can be, for example, fused into a plant transformation vector suitable for the stable incorporation of the heterologous polynucleotide into the genome of the plant cell. Typically, the stably transformed plant cell will be regenerated into a transformed plant that comprises in its genome the heterologous polynucleotide. Such a stably transformed plant is capable of transmitting the heterologous polynucleotide to progeny plants in subsequent generations via sexual and/or asexual reproduction. Plant transformation vectors, methods for stably transforming plants with an introduced heterologous polynucleotide and methods for plant regeneration from transformed plant cells and tissues are generally known in the art for both monocotyledonous and dicotyledonous plants or described elsewhere herein.

In other embodiments in which it is not desired to stably incorporate the heterologous polynucleotide in the genome of the plant, transient transformation methods can be utilized to introduce the heterologous polynucleotide into one or more plant cells of a plant. Such transient transformation methods include, for example, viral-based methods which involve the use of viral particles or at least viral nucleic acids. Generally, such viral-based methods involve constructing a modified viral nucleic acid comprising a heterologous polynucleotide of the disclosure operably linked to the viral nucleic acid and then contacting the plant either with a modified virus comprising the modified viral nucleic acid or with the viral nucleic acid or with the modified viral nucleic acid itself. The modified virus and/or modified viral nucleic acids can be applied to the plant or part thereof, for example, in accordance with conventional methods used in agriculture, for example, by spraying, irrigation, dusting, or the like. The modified virus and/or modified viral nucleic acids can be applied in the form of directly sprayable solutions, powders, suspensions or dispersions, emulsions, oil dispersions, pastes, dustable products, materials for spreading, or granules, by means of spraying, atomizing, dusting, spreading or pouring. It is recognized that it may be desirable to prepare formulations comprising the modified virus and/or modified viral nucleic acids before applying to the plant or part or parts thereof. Methods for making pesticidal formulations are generally known in the art or described elsewhere herein.

The present disclosure provides nucleic acid molecules comprising protease inhibitor nucleotide sequences. Preferably, such nucleic acid molecules are capable of conferring upon a host plant, particularly a potato host plant enhanced resistance to a plant disease caused by at least one bacterial pathogen. Thus, such nucleic acid molecules find use in limiting a plant disease caused by at least one bacterial pathogen in agricultural production. The nucleic acid molecules of the present disclosure include, but are not limited to, nucleic acid molecules comprising at least one protease inhibitor nucleotide sequence disclosed herein but also additional orthologs and other variants of the protease inhibitor nucleotide sequences that are capable of conferring to a plant resistance to a plant disease caused by at least one bacterial pathogen. Methods are known in the art or otherwise disclosed herein for determining resistance of a plant to a plant disease caused by at least one bacterial pathogen, including, for example, the virulence assay utilizing potato tubers that is described elsewhere herein.

The present disclosure further provides plants and cells thereof, particularly potato plants and cells thereof, comprising g18987, g28531, g39249, g40384, and/or g6571, and/or alleles, homologs, and other naturally and non-naturally occurring variants of such protease inhibitor genes, and that are produced by methods that do not involve the introduction of recombinant DNA into the plant or a cell thereof. Such methods can comprise, for example, interspecific hybridizations involving two or more different plant species. In preferred embodiments, the plants are solanaceous plants.

Additionally provided are methods for introducing at least one protease inhibitor gene of present disclosure into a plant, particularly a potato plant, lacking in its genome the at least one protease inhibitor gene. The protease inhibitor genes of the present disclosure include, for example, g18987, g28531, g39249, g40384, and g6571, and alleles, homologs, and other naturally and non-naturally occurring variants of such protease inhibitor genes, and/or protease inhibitor genes comprising a nucleotide sequence set forth in SEQ ID NOs: 6-15 and/or encoding protease inhibitor protein comprising an amino acid sequence set forth in SEQ ID NOs: 1-5. The methods comprise crossing (i.e. cross-pollinating) a first plant comprising in its genome at least one copy of a protease inhibitor gene of present disclosure with a second plant lacking in its genome the protease inhibitor gene. The first and second plants can be the same species or can be different species. In a preferred embodiment, the first and second plants are solanaceous plants. For example, the first plant can be Solanum chacoense and the second plant can be Solanum tuberosum. Such a crossing of a first species of a plant to a second species of a plant is known as an interspecific hybridization and can be used to introgress a gene or genes of interest (e.g. a protease inhibitor) from one species into a related species lacking the gene or genes of interest and typically involves multiple generations of backcrossing of the progeny with the related species and selection at each generation of progeny comprising the gene or genes of interest. Such interspecific hybridization, introgression, and backcrossing methods are well known in the art and can be used in the methods of the present invention. See “Principals of Cultivar Development.” Fehr, 1993. Macmillan Publishing Company, New York; and “Fundamentals of Plant Genetics and Breeding,” Welsh, 1981, John Wiley & Sons, Inc., New York.

In methods of the present disclosure for introducing at least one protease inhibitor gene of present disclosure into a plant lacking in its genome the at least one protease inhibitor gene, either the first plant or the second plant can be the pollen donor plant. For example, if the first plant is the pollen donor plant, then the second plant is the pollen-recipient plant. Likewise, if the second plant is the pollen donor plant, then the first plant is the pollen-recipient plant. Following the crossing, the pollen-recipient plant is grown under conditions favorable for the growth and development of the plant and for a sufficient period of time for seed to mature or to achieve an otherwise desirable growth stage for use in a subsequent in vitro germination procedure such as, for example, embryo rescue that is described below. The seed can then be harvested and those seed comprising the protease inhibitor gene(s) identified by any method known in the art including, for example, the methods for identifying a potato plant that displays newly conferred or enhanced resistance to a plant disease caused by at least one bacterial pathogen that are described elsewhere herein. In certain embodiments, the first plant is a Solanum chacoense plant comprising the protease inhibitor gene(s) and the second plant is Solanum tuberosum plant lacking the protease inhibitor gene(s). In certain embodiments, the first plant is a Solanum chacoense plant comprising the protease inhibitor gene(s) or other solanaceous plant species comprising in its genome the protease inhibitor gene(s) and the second plant is a solanaceous plant species other than Solanum chacoense.

It is recognized, however, that in certain embodiments involving interspecific hybridizations, it may be advantageous to harvest the seed resulting from such interspecific hybridizations at an immature growth stage and then to germinate the immature seeds in culture (i.e. in vitro), whereby the seeds are allowed germinate in culture using methods known in art as “embryo rescue” methods. See Reed (2005) “Embryo Rescue,” in Plant Development and Biotechnology, Trigiano and Gray, eds. (PDF). CRC Press, Boca Raton, pp. 235-239; and Sharma et al. (1996) Euphytica 89:325-337. It is further recognized that embryo rescue methods are typically used when mature seeds produced by an interspecific cross display little or no germination, whereby few or no interspecific hybrid plants are produced.

The methods of the present disclosure find use in producing plants with enhanced resistance to a plant disease caused by at least one bacterial pathogen. Typically, the methods of the present disclosure will enhance or increase the resistance of the subject plant to the plant disease by at least 25%, 50%, 75%, 100%, 150%, 200%, 250%, 500% or more when compared to the resistance of a control plant to the same bacterial pathogen(s). Unless stated otherwise or apparent from the context of a use, a control plant is a plant that does not comprise the heterologous polynucleotide and/or protease inhibitor nucleotide sequence of the present disclosure. Preferably, the control plant is essentially identical (e.g. same species, subspecies, and variety) to the plant comprising the heterologous polynucleotide of the present disclosure except the control does not comprise the heterologous polynucleotide or protease inhibitor nucleotide sequence. In some embodiments, the control will comprise a heterologous polynucleotide but not comprise the one or more protease inhibitor nucleotide sequences that are in a heterologous polynucleotide of the present invention.

Additionally, the present disclosure provides transformed plants, seeds, and plant cells produced by the methods of present disclosure and/or comprising a heterologous polynucleotide of the present disclosure. Also provided are progeny plants and seeds thereof comprising a heterologous polynucleotide of the present disclosure. The present disclosure also provides fruits, seeds, tubers, leaves, stems, roots, and other plant parts produced by the transformed plants and/or progeny plants of the disclosure as well as food products and other agricultural products comprising, or produced or derived from, the plants or any part or parts thereof including, but not limited to, fruits, tubers, leaves, stems, roots, and seed. Other agricultural products include, for example, food and industrial starch products produced from potato tubers. It is recognized that such food products can be consumed or used by humans and other animals including, but not limited to, pets (e.g. dogs and cats), livestock (e.g. pigs, cows, chickens, turkeys, and ducks), and animals produced in freshwater and marine aquaculture systems (e.g. fish, shrimp, prawns, crayfish, and lobsters).

Unless expressly stated or apparent from the context of usage, the methods and compositions of the present disclosure can be used with any plant species including, for example, monocotyledonous plants, dicotyledonous plants, and conifers. Examples of plant species of interest include, but are not limited to, corn (Zea mays), Brassica sp. (e.g. B. napus, B. rapa, B. juncea), particularly those Brassica species useful as sources of seed oil, alfalfa (Medicago sativa), rice (Oryza sativa), rye (Secale cereale), triticale (×Triticosecale or Triticum×Secale) sorghum (Sorghum bicolor, Sorghum vulgare), teff (Eragrostis tef), millet (e.g. pearl millet (Pennisetum glaucum), proso millet (Panicum miliaceum), foxtail millet (Setaria italica), finger millet (Eleusine coracana)), switchgrass (Panicum virgatum), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossvpium hirsutum), strawberry (e.g. Fragaria ananassa, Fragaria vesca, Fragaria moschata, Fragaria virginiana, Fragaria chiloensis), sweet potato (Ipomoea batatus), yam (Dioscorea spp., D. rotundata, D. cayenensis, D. alata, D. polystachwya, D. bulbifera, D. esculenta, D. dumetorum, D. trifida), cassava (Manihot esculenta), coffee (Coffea spp.), coconut (Cocos nucifera), oil palm (e.g. Elaeis guineensis, Elaeis oleifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), date (Phoenix dactylifera), cultivated forms of Beta vulgaris (sugar beets, garden beets, chard or spinach beet, mangel-wurzel or fodder beet), sugarcane (Saccharum spp.), oat (Avena sativa), barley (Hordeum vulgare), cannabis (Cannabis sativa, C. indica. C. ruderalis), poplar (Populus spp.), eucalyptus (Eucalyptus spp.), Arabidopsis thaliana. Arabidopsis rhizogenes, Nicotiana benthamiana, Brachypodium distachyon vegetables, ornamentals, and conifers and other trees. In specific embodiments, plants of the present invention are crop plants (e.g. potato, tobacco, tomato, maize, sorghum, wheat, millet, rice, barley, oats, sugarcane, alfalfa, soybean, peanut, sunflower, cotton, safflower, Brassica spp., lettuce, strawberry, apple, citrus, etc.).

Vegetables include tomatoes (Solanum lycopersicum), eggplant (also known as “aubergine” or “brinjal”)(Solanum melongena), pepper (Capsicum annuum), lettuce (e.g. Lactuca sativa), green beans (Phaseolus vulgaris), lima beans (Phaseolus limensis), peas (Lathyrus spp.), chickpeas (Cicer arietinum), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Fruit trees and related plants include, for example, apples, pears, peaches, plums, oranges, grapefruits, limes, pomelos, palms, and bananas. Nut trees and related plants include, for example, almonds, cashews, walnuts, pistachios, macadamia nuts, filberts, hazelnuts, and pecans.

In specific embodiments, the plants of the present disclosure are crop plants such as, for example, maize (corn), soybean, wheat, rice, cotton, alfalfa, sunflower, canola (Brassica spp., particularly Brassica napus, Brassica rapa, Brassica juncea), rapeseed (Brassica napus), sorghum, millet, barley, triticale, safflower, peanut, sugarcane, tobacco, potato, tomato, and pepper.

Preferred plants of the disclosure are solanaceous plants. As used herein, the term “solanaceous plant” refers to a plant that is a member of the Solanaceae family. Such solanaceous plants include, for example, domesticated and non-domesticated members of Solanaceae family. Solanaceous plants of the present disclosure include, but are not limited to, potato (Solanum tuberosum), eggplant (Solanum melongena), petunia (Petunia spp., e.g. Petunia×hybrida or Petunia hybrida), tomatillo (Physalis philadelphica), Cape gooseberry (Physalis peruviana), Physalis sp., woody nightshade (Solanum dulcamara), garden huckleberry (Solanum scabrum), gboma eggplant (Solanum macrocarpon), pepper (Capsicum spp; e.g. Capsicum annuum, C. baccaltum, C. chinense, C. frutescens, C. pubescens, and the like), tomato (Solanum lycopersicum), tobacco (Nicotiana spp., e.g. N. tabacum. N. benthamiana), Solanum americanum, Solanum nigrescens Solanum demissum, Solanum stolonferum, Solanum papita, Solanum bulbocastanum, Solanum edinense, Solanum schenckii, Solanum hjertingii, Solanum venturi, Solanum mochiquense, Solanum chacoense, and Solanum pimpinellifolium. In preferred embodiments of the methods and compositions of the present disclosure, the solanaceous plants are solanaceous plants grown in agriculture including, but not limited to, potato, tomato, tomatillo, Cape gooseberry, eggplant, pepper, tobacco, and petunia. In more preferred embodiments, the solanaceous plant is potato. In certain other embodiments of the methods and compositions disclosed herein, the preferred solanaceous plants are all solanaceous plants except for Solanum chacoense. In yet other embodiments of the methods and compositions disclosed herein, the preferred plants are all plants except for Solanum chacoense.

The term “solanaceous plant” is intended to encompass solanaceous plants at any stage of maturity or development, as well as any cells, tissues or organs (plant parts) taken or derived from any such plant unless otherwise clearly indicated by context. Solanaceous plant parts include, but are not limited to, fruits, stems, tubers, roots, flowers, ovules, stamens, leaves, embryos, meristematic regions, callus tissue, anther cultures, gametophytes, sporophytes, pollen, microspores, protoplasts, and the like. As used herein, the term “tuber” is intended to mean a whole tuber or any part thereof such as, for example, a slice or a portion of potato tuber comprising one or more buds (i.e. “eyes”) suitable for planting in a field to produce a potato plant. The present disclosure also includes seeds produced by the solanaceous plants of the present disclosure.

“Potato” or “cultivated potato” refers herein to plants of the species Solanum tuberosum, and parts of such plants, bred by humans for food and having good agronomic characteristics. This includes any cultivated potato, such as breeding lines (e.g. backcross lines, inbred lines), cultivars and varieties (open pollinated or hybrids). Wild relatives of potato, such as Solanum chacoense or Solanum jamesii, are not encompassed by this definition.

The composition and methods of the present disclosure find use in producing plants with enhanced resistance to at least one bacterial pathogen. Bacterial pathogens causing damage to plants or to a part of a plant include inter alia Actinobacteria and Proteobacteria and are selected from the families of the Burkholderiaceae, Xanthomonadaceae, Pseudomonadaceae, Erwiniaceae, Microbacteriaceae, Pectobacteriaceae, and Rhizobiaceae.

Pectobacteriaceae is a family of Gram-negative bacteria which includes Pectobacterium spp. and Dickeya spp. The family is a member of the order Enterobacterales in the class Gammaproteobacteria of the phylum Proteobacteria. Pectobacterium spp. include, but are not limited to, Pectobacterium actinidiae, Pectobacterium aquaticum, Pectobacterium aroidearum, Pectobacterium atrosepticum, Pectobacterium betavasculorum, Pectobacterium brasiliense, Pectobacterium cacticida, Pectobacterium carotovorum, Pectobacterium fontis, Pectobacterium odoriferum, Pectobacterium parmentieri, Pectobacterium parvum, Pectobacterium peruviense, Pectobacterium polaris, Pectobacterium polonicum, Pectobacterium punjabense, Pectobacterium quasiaquaticum, Pectobacterium versatile, Pectobacterium wasabiae, and Pectobacterium zantedeschiae. Dickeya spp. include, but are not limited to, Dickeya aquatica, Dickeya chrysanthemi, Dickeya dadantii, Dickeya dianthicola, Dickeya fangzhongdai, Dickeya lacustris, Dickeya oryzae, Dickeya parazeae, Dickeya poaceiphila, Dickeya solani, Dickeya undicola, and Dickeya zeae.

In certain embodiments, the bacterial pathogen is capable of causing a soft rot on at least one plant, in particular a soft rot or blackleg of potato. Bacterial soft rots damage plant parts such as fruits, tubers, stems, and bulbs of plants in nearly every plant family. Soft rots commonly affect vegetables such as potato, carrot, onion, beet, tomato, cucurbits (e.g., cucumbers, melons, squash, pumpkins), and cruciferous crops (e.g., cabbage, cauliflower, bok choy). Soft rots are caused by several bacterial species, most commonly Pectobacterium spp., Dickeya spp., Erwinia spp., and Pseudomonas spp.

In one embodiment, the nucleotide sequences encoding protease inhibitor proteins have at least about 75%, 80%, 85%, 90%, 91%, 92%, 93%, 94%, 95%, 96%, 97%, 98%, 99% or more sequence identity to the entire nucleotide sequence set forth in at least one of SEQ ID NOs: 6-15 or to a fragment thereof. In another embodiment, the nucleotide sequences encoding protease inhibitor proteins have at least about 75%, 80%, 85%, 90%, 91%, 92%, 93%, 94%, 95%, 96%, 97%, 98%, 99% or more sequence identity to the entire nucleotide sequence set forth in at least one of SEQ ID NOs: 6-15 or to a fragment thereof.

The present disclosure encompasses isolated or substantially purified polynucleotide (also referred to herein as “nucleic acid molecule”, “nucleic acid” and the like) or protein (also referred to herein as “polypeptide”) compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e. sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

Fragments and variants of the disclosed polynucleotides and proteins encoded thereby are also encompassed by the present disclosure. By “fragment” is intended a portion of the polynucleotide or a portion of the amino acid sequence and hence protein encoded thereby. Fragments of polynucleotides comprising coding sequences may encode protein fragments that retain biological activity of the full-length or native protein. Alternatively, fragments of a polynucleotide that are useful as hybridization probes generally do not encode proteins that retain biological activity or do not retain promoter activity. Thus, fragments of a nucleotide sequence may range from at least about 20 nucleotides, about 50 nucleotides, about 100 nucleotides, and up to the full-length polynucleotide of the disclosure.

In certain embodiments, the fragments and variants of the disclosed polynucleotides and proteins encoded thereby are those that are capable of conferring to a plant resistance to a plant disease caused by at least one bacterial pathogen. Preferably, a polynucleotide comprising a fragment of a native protease inhibitor polynucleotide of the present disclosure is capable of conferring resistance to a plant disease caused by at least one race of at least one bacterial pathogen to a plant comprising the polynucleotide. Likewise, a protein or polypeptide comprising a native protease inhibitor protein of the present disclosure is preferably capable of conferring resistance to a plant disease caused by at least one race of at least one bacterial pathogen to a plant comprising the protein or polypeptide.

Polynucleotides that are fragments of a native protease inhibitor polynucleotide comprise at least 16, 20, 50, 75, 100, 125, 150, 175, 200, 250, 300, 350, 400, 450, 500, 600, 700, 800, 900, 1000, 1500, 2000, 3000, 4000, 5000, 6000, 7000, 8000, or 9000 contiguous nucleotides, or up to the number of nucleotides present in a full-length protease inhibitor polynucleotide disclosed herein.

“Variants” is intended to mean substantially similar sequences. For polynucleotides, a variant comprises a polynucleotide having deletions (i.e. truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the protease inhibitor proteins of the disclosure. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode an protease inhibitor protein of the disclosure. Generally, variants of a particular polynucleotide of the disclosure will have at least about 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. In certain embodiments, variants of a particular polynucleotide of the invention will have at least about 75%, 80%, 85%, 90%, 91%, 92%, 93%, 94%, 95%, 96%, 97%, 98%, 99% or more sequence identity to at least one nucleotide sequence selected from SEQ ID NOs: 6-15, and optionally comprise a non-naturally occurring nucleotide sequence that differs from the nucleotide sequence set forth in SEQ ID NOs: 6-15 by at least one nucleotide modification, wherein the at least one nucleotide modification comprises the substitution of at least one nucleotide, the addition of at least one nucleotide, or the deletion of at least one nucleotide. It is understood that the addition of at least one nucleotide can be the addition of one or more nucleotides within a nucleotide sequence of the present disclosure (e.g. SEQ ID NOs: 6-15), the addition of one or more nucleotides to the 5′ end of a nucleotide sequence of the present disclosure, and/or the addition of one or more nucleotides to the 3′ end of a nucleotide sequence of the present disclosure.

Variants of a particular polynucleotide of the disclosure (i.e. the reference polynucleotide) can also be evaluated by comparison of the percent sequence identity between the polypeptide encoded by a variant polynucleotide and the polypeptide encoded by the reference polynucleotide. Thus, for example, a polynucleotide that encodes a polypeptide with a given percent sequence identity to at least one polypeptide having the amino acid sequence selected from the group consisting of SEQ ID NOs: 1-5 is disclosed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 60%, 65%, 70%, 75%, 80%, 85%, 90%, 91%, 92%, 93%, 94%, 95%, 96%, 97%, 98%, 99% or more sequence identity. In certain embodiments of the invention, variants of a particular polypeptide of the invention will have at least about 60%, 65%, 70%, 75%, 80%, 85%, 90%, 91%, 92%, 93%, 94%, 95%, 96%, 97%, 98%, 99% or more sequence identity to at least one of the amino acid sequences set forth in SEQ ID NOs: 1-5, and optionally comprises a non-naturally occurring amino acid sequence that differs from at least one amino acid sequence selected from SEQ ID NOs: 1-5 by at least one amino acid modification, wherein the at least one amino acid modification comprises the substitution of at least one amino acid, the addition of at least one amino acid, or the deletion of at least one amino acid. It is understood that the addition of at least one amino acid can be the addition of one or more amino acids within an amino acid sequence of the present disclosure (e.g. SEQ ID NOs: 1-5), the addition of one or more amino acids to the N-terminal end of an amino acid sequence of the present disclosure, and/or the addition of one or more amino acids to the C-terminal end of an amino acid sequence of the present disclosure.

“Variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Such variants may result from, for example, genetic polymorphism or from human manipulation. Biologically active variants of an R protein will have at least about 75%, 80%, 85%, 90%, 91%, 92%, 93%, 94%, 95%, 96%, 97%, 98%, 99% or more sequence identity to the amino acid sequence for the native protein (e.g. the amino acid sequence set forth in SEQ ID NO: 1, 2, 3, 4, or 5) as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

The proteins of the disclosure may be altered in various ways including amino acid substitutions, deletions, truncations, and insertions. Methods for such manipulations are generally known in the art. Methods for mutagenesis and polynucleotide alterations are well known in the art. See, for example, Kunkel (1985) Proc. Natl. Acad. Sci. USA 82:488-492; Kunkel et al. (1987) Methods in Enzymollette. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington. D.C.), herein incorporated by reference. Conservative substitutions, such as exchanging one amino acid with another having similar properties, may be optimal.

Thus, the genes and polynucleotides of the disclosure include both the naturally occurring sequences as well as mutant and other variant forms. Likewise, the proteins of the disclosure encompass naturally occurring proteins as well as variations and modified forms thereof. More preferably, such variants confer to a plant or part thereof comprising the variant enhanced resistance a plant disease caused by at least one bacterial pathogen. In some embodiments, the mutations that will be made in the DNA encoding the variant will not place the sequence out of reading frame. Optimally, the mutations will not create complementary regions that could produce secondary mRNA structure. See, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by assays that are disclosed herein below.

Variant polynucleotides and proteins also encompass sequences and proteins derived from a mutagenic and recombinogenic procedure such as DNA shuffling. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The polynucleotides of the disclosure can be used to isolate corresponding sequences from other organisms, particularly other plants. In this manner, methods such as PCR, hybridization, and the like can be used to identify such sequences based on their sequence homology to the sequences set forth herein. Sequences isolated based on their sequence identity to the entire sequences set forth herein or to variants and fragments thereof are encompassed by the present invention. Such sequences include sequences that are orthologs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 60%, 70%, 75%, 80%, 85%, 90%, 91%, 92%, 93%, 94%, 95%, 96%, 97%, 98%, 99%, or greater sequence identity. Functions of orthologs are often highly conserved among species. Thus, isolated polynucleotides that encode protease inhibitor proteins having at least 60% amino acid sequence identity to a full-length amino acid sequence of at least one of the protease inhibitor proteins disclosed herein or otherwise known in the art, or to variants or fragments thereof, are encompassed by the present disclosure.

In one embodiment, the orthologs of the present disclosure have coding sequences comprising at least 80%, 85%, 90%, 91%, 92%, 93%, 94%, 95%, 96%, 97%, 98%, 99%, or greater nucleotide sequence identity to at least one nucleotide sequence selected from the nucleotide sequences set forth in SEQ ID NOs: 6-15 and/or encode proteins comprising least 80%, 85%, 90%, 91%, 92%, 93%, 94%, 95%, 96%, 97%, 98%, 99%, or greater amino acid sequence identity to at least one amino acid sequence selected from the amino acid sequences set forth in SEQ ID NOs: 1-5.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known polynucleotide is used as a probe that selectively hybridizes to other corresponding polynucleotides present in a population of cloned genomic DNA fragments or cDNA fragments (i.e. genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the polynucleotides of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, New York).

For example, an entire polynucleotide disclosed herein, or one or more portions thereof, may be used as a probe capable of specifically hybridizing to corresponding polynucleotide and messenger RNAs. To achieve specific hybridization under a variety of conditions, such probes include sequences that are unique among the sequence of the gene or cDNA of interest sequences and are optimally at least about 10 nucleotides in length, and most optimally at least about 20 nucleotides in length. Such probes may be used to amplify corresponding polynucleotides for the particular gene of interest from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, New York). An extensive guide to the hybridization of nucleic acids is found in Tijssen (1993) Laboratory Techniques in Biochemistry and Molecular Biology Hybridization with Nucleic Acid Probes, Part 1, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

It is recognized that the protease inhibitor protein coding sequences of the present disclosure encompass polynucleotide molecules comprising a nucleotide sequence that is sufficiently identical to the nucleotide sequence of any one or more of SEQ ID NOs: 1-5. The term “sufficiently identical” is used herein to refer to a first amino acid or nucleotide sequence that contains a sufficient or minimum number of identical or equivalent (e.g. with a similar side chain) amino acid residues or nucleotides to a second amino acid or nucleotide sequence such that the first and second amino acid or nucleotide sequences have a common structural domain and/or common functional activity. For example, amino acid or nucleotide sequences that contain a common structural domain having at least about 45%, 55%, or 65% identity, preferably 75% identity, more preferably 85%, 90%, 95%, 96%, 97%, 98% or 99% identity are defined herein as sufficiently identical.

To determine the percent identity of two amino acid sequences or of two nucleic acids, the sequences are aligned for optimal comparison purposes. The percent identity between the two sequences is a function of the number of identical positions shared by the sequences (i.e. percent identity=number of identical positions/total number of positions (e.g. overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A preferred, nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences homologous to the polynucleotide molecules of the invention. BLAST protein searches can be performed with the XBLAST program, score=50, wordlength=3, to obtain amino acid sequences homologous to protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g. XBLAST and NBLAST) can be used. BLAST, Gapped BLAST, and PSI-Blast, XBLAST and NBLAST are available on the World Wide Web at ncbi.nlm.nih.gov. Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM 120 weight residue table, a gap length penalty of 12, and a gap penalty of 4 can be used. Alignment may also be performed manually by inspection.

Unless otherwise stated, sequence identity/similarity values provided herein refer to the value obtained using the full-length sequences of the invention and using multiple alignment by mean of the algorithm Clustal W (Nucleic Acid Research, 22(22):4673-4680, 1994) using the program AlignX included in the software package Vector NTI Suite Version 7 (InforMax, Inc., Bethesda, Md., USA) using the default parameters; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by CLUSTALW (Version 1.83) using default parameters (available at the European Bioinformatics Institute website on the World Wide Web at ebi.ac.uk/Tools/clustalw/index).

The use of the term “polynucleotide” is not intended to limit the present disclosure to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the disclosure also encompass all forms of sequences including, but not limited to, single-stranded forms, double-stranded forms, hairpins, stem-and-loop structures, and the like.

The heterologous polynucleotides or polynucleotide constructs comprising protease inhibitor protein coding regions can be provided in expression cassettes for expression in the plant or other organism or non-human host cell of interest. The cassette will include 5′ and 3′ regulatory sequences operably linked to the protease inhibitor protein coding region. “Operably linked” is intended to mean a functional linkage between two or more elements. For example, an operable linkage between a polynucleotide or gene of interest and a regulatory sequence (i.e. a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be co-transformed into the organism. Alternatively, the additional gene(s) can be provided on multiple expression cassettes. Such an expression cassette is provided with a plurality of restriction sites and/or recombination sites for insertion of the R protein coding region to be under the transcriptional regulation of the regulatory regions. The expression cassette may additionally contain selectable marker genes.

The expression cassette can include in the 5′-3′ direction of transcription, a transcriptional and translational initiation region (i.e. a promoter), a protease inhibitor protein coding region of the disclosure, and a transcriptional and translational termination region (i.e. termination region) functional in plants or other organism or non-human host cell. The regulatory regions (i.e. promoters, transcriptional regulatory regions, and translational termination regions) and/or the protease inhibitor protein coding region of the disclosure may be native/analogous to the host cell or to each other. Alternatively, the regulatory regions and/or the protease inhibitor protein coding region of the invention may be heterologous to the host cell or to each other.

As used herein, “heterologous” in reference to a nucleic acid molecule, polynucleotide, nucleotide sequence, or polynucleotide construct is a nucleic acid molecule, polynucleotide, nucleotide sequence, or polynucleotide construct that originates from a foreign species, or, if from the same species, is modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

As used herein, a “native gene” is intended to mean a gene that is a naturally-occurring gene in its natural or native position in the genome of a plant. Such a native gene has not been genetically engineered or otherwise modified in nucleotide sequence and/or position in the genome the plant through human intervention, nor has such a native gene been introduced into the genome of the plant via artificial methods such as, for example, plant transformation.

As used herein, a “non-native gene” is intended to mean a gene that has been introduced into a plant by artificial means and/or comprises a nucleotide sequence that is not naturally occurring in the plant. Non-native genes include, for example, a gene (e.g. a protease inhibitor gene) that is introduced into the plant by a plant transformation method. Additionally, when a native gene in the genome of a plant is modified, for example by a genome-editing method, to comprise a nucleotide sequence that is different (i.e. non-identical) from the nucleotide sequence of native gene, the modified gene is a non-native gene.

The present disclosure provides host cells comprising at least one of the nucleic acid molecules, expression cassettes, and vectors of the present disclosure. In preferred embodiments, a host cell is a plant cell. In other embodiments, a host cell is selected from a bacterium, a fungal cell, and an animal cell. In certain embodiments, a host cell is non-human animal cell. However, in some other embodiments, the host cell is an in-vitro cultured human cell.

While it may be optimal to express the protease inhibitor protein using heterologous promoters, the native promoter of the corresponding protease inhibitor gene may be used.

The termination region may be native with the transcriptional initiation region, may be native with the operably linked protease inhibitor protein coding region of interest, may be native with the plant host, or may be derived from another source (i.e. foreign or heterologous to the promoter, the protease inhibitor protein of interest, and/or the plant host), or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991)(ell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); poty virus leaders, for example, TEV leader (Tobacco Etch Virus)(Gallie et al. (1995) Gene 165(2):233-238), MDMV leader (Maize Dwarf Mosaic Virus)(Virology 154:9-20), and human immunoglobulin heavy-chain binding protein (BiP)(Macejak et al. (1991) Nature 353:90-94); untranslated leader from the coat protein mRNA of alfalfa mosaic virus (AMV RNA 4)(Jobling et al. (1987) Nature 325:622-625); tobacco mosaic virus leader (TMV)(Gallie et al. (1989) in Molecular Biology of RNA, ed. Cech (Liss, New York), pp. 237-256); and maize chlorotic mottle virus leader (MCMV)(Lommel et al. (1991) Virology 81:382-385). See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g. transitions and transversions, may be involved.

A number of promoters can be used in the practice of the embodiments. The promoters can be selected based on the desired outcome. The nucleic acids can be combined with constitutive, tissue-preferred, or other promoters for expression in plants. Such constitutive promoters include, for example, the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced expression of the protease inhibitor protein coding sequences within a particular plant tissue. Such tissue-preferred promoters include, but are not limited to, leaf-preferred promoters, root-preferred promoters, seed-preferred promoters, and stem-preferred promoters. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112 (2): 525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23 (6): 1129-1138; Matsuoka et al. (1993) Proc Natl. Acad. Sci. USA 90 (20): 9586-9590; and Guevara-Garcia et al. (1993) Plant J. 4 (3): 495-505. Such promoters can be modified, if necessary, for weak expression.

In certain embodiments, it will be beneficial to express the gene from an inducible promoter, particularly from a pathogen-inducible promoter. Such promoters include those from pathogenesis-related proteins (PR proteins), which are induced following infection by a pathogen; e.g. PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Such inducible promoters include the maize PRms gene promoter, whose expression is induced by the pathogen Fusarium moniliforme (see, for example, Cordero et al. (1992) Physiol. Mol. Plant Path. 41:189-200).

Additionally, as pathogens find entry into plants through wounds or insect damage, a wound-inducible promoter may be used in the heterologous polynucleotides of the disclosure. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6 (2): 141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14 (2): 247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP)(Su et al. (2004) Biotechnol Bioeng 85:610-9 and Fetter et al. (2004) Plant Cell 16:215-28), cyan florescent protein (CYP)(Bolte et al. (2004). J. Cell Science 117:943-54 and Kato et al. (2002) Plant Physiol 129:913-42), and yellow florescent protein (Phi YFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschmidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not intended to be limiting. Any selectable marker gene can be used.

Numerous plant transformation vectors and methods for transforming plants are available. See, for example, An, G. et al. (1986) Plant Pysiol., 81:301-305; Fry, J., et al. (1987) Plant Cell Rep. 6:321-325; Block. M. (1988) Theor. Appl Genet. 76:767-774; Hinchee, et al. (1990) Stadler. Genet. Symp. 203212.203-212; Cousins, et al. (1991) Aust. J. Plant Physiol. 18:481-494; Chee, P. P. and Slightom, J. L. (1992) Gene. 118:255-260; Christou, et al. (1992) Trends. Biotechnol. 10:239-246; D'Halluin, et al. (1992) Bio/Technol. 10:309-314; Dhir, et al. (1992) Plant Physiol. 99:81-88; Casas et al. (1993) Proc. Nat. Acad Sci. USA 90:11212-11216; Christou, P. (1993) In Vitro Cell. Dev. Biol.-Plant; 29P: 119-124; Davies, et al. (1993) Plant Cell Rep. 12:180-183; Dong, J. A. and Mchughen, A. (1993) Plant Sci. 91:139-148; Franklin. C. I. and Trieu, T. N. (1993) Plant. Physiol. 102:167; Golovkin, et al. (1993) Plant Sci. 90:41-52; Guo Chin Sci. Bull. 38:2072-2078; Asano, et al. (1994) Plant Cell Rep. 13; Ayeres N. M. and Park, W. D. (1994) Crit. Rev. Plant. Sci. 13:219-239; Barcelo, et al. (1994) Plant. J. 5:583-592; Becker, et al. (1994) Plant. J. 5:299-307; Borkowska et al. (1994) Acta. Physiol Plant. 16:225-230; Christou, P. (1994) Agro. Food. Ind. Hi Tech. 5:17-27; Eapen et al. (1994) Plant Cell Rep. 13:582-586; Hartman, et al. (1994) Bio-Technology 12:919923; Ritala, et al. (1994) Plant. Mol. Biol. 24:317-325; and Wan, Y. C. and Lemaux, P. G. (1994) Plant Physiol. 104:3748.

The methods of the disclosure involve introducing a heterologous polynucleotide or polynucleotide construct into a plant. By “introducing” is intended presenting to the plant the heterologous polynucleotide or polynucleotide construct in such a manner that the construct gains access to the interior of a cell of the plant. The methods of the disclosure do not depend on a particular method for introducing a heterologous polynucleotide or polynucleotide construct to a plant, only that the heterologous polynucleotide or polynucleotide construct gains access to the interior of at least one cell of the plant. Methods for introducing heterologous polynucleotides or polynucleotide constructs into plants are known in the art including, but not limited to, stable transformation methods, transient transformation methods, and virus-mediated methods.

By “stable transformation” is intended that the heterologous polynucleotide or polynucleotide construct introduced into a plant integrates into the genome of the plant and is capable of being inherited by progeny thereof. By “transient transformation” is intended that a heterologous polynucleotide or polynucleotide construct introduced into a plant does not integrate into the genome of the plant. It is recognized that stable and transient transformation methods comprise introducing one or more nucleic acid molecules (e.g. DNA), particularly one or more recombinant nucleic acid molecules (e.g. recombinant DNA) into a plant, plant cell, or other host cell or organism.

For the transformation of plants and plant cells, the nucleotide sequences of the disclosure are inserted using standard techniques into any vector known in the art that is suitable for expression of the nucleotide sequences in a plant or plant cell. The selection of the vector depends on the preferred transformation technique and the target plant species to be transformed.

Methodologies for constructing plant expression cassettes and introducing foreign nucleic acids into plants are generally known in the art and have been previously described. For example, foreign DNA can be introduced into plants, using tumor-inducing (Ti) plasmid vectors. Other methods utilized for foreign DNA delivery involve the use of PEG mediated protoplast transformation, electroporation, microinjection whiskers, and biolistics or microprojectile bombardment for direct DNA uptake. Such methods are known in the art. (U.S. Pat. No. 5,405,765 to Vasil et al.; Bilang et al. (1991) Gene 100:247-250; Scheid et al., (1991) Mol. Gen. Genet., 228:104-112; Guerche et al., (1987) Plant Science 52:111-116; Neuhause et al., (1987) Theor. Appl Genet. 75:30-36; Klein et al., (1987) Nature 327:70-73; Howell et al., (1980) Science 208:1265; Horsch et al., (1985) Science 227:1229-1231; DeBlock et al., (1989) Plant Physiology 91:694-701; Methods for Plant Molecular Biology (Weissbach and Weissbach, eds.) Academic Press, Inc. (1988) and Methods in Plant Molecular Biology (Schuler and Zielinski, eds.) Academic Press, Inc. (1989). The method of transformation depends upon the plant cell to be transformed, stability of vectors used, expression level of gene products and other parameters.

Other suitable methods of introducing nucleotide sequences into plant cells and subsequent insertion into the plant genome include microinjection as Crossway et al. (1986) Biotechniques 4:320-334, electroporation as described by Riggs et al. (1986) Proc. Natl. Acad. Sci. USA 83:5602-5606, Agrobacterium-mediated transformation as described by Townsend et al., U.S. Pat. No. 5,563,055, Zhao et al., U.S. Pat. No. 5,981,840, direct gene transfer as described by Paszkowski et al. (1984) EMBO J. 3:2717-2722, and ballistic particle acceleration as described in, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lecl transformation (WO 00/28058). Also see, Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin)(maize); Klein et al. (1988) Plant Physiol. 91:440-444 (maize); Fromm et al. (1990) Biotechnology 8:833-839 (maize); HooykaasVan Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The polynucleotides of the disclosure may be introduced into plants by contacting plants with a virus or viral nucleic acids. Generally, such methods involve incorporating a heterologous polynucleotide or polynucleotide construct of the disclosure within a viral DNA or RNA molecule. Further, it is recognized that promoters of the disclosure also encompass promoters utilized for transcription by viral RNA polymerases. Methods for introducing polynucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

If desired, the modified viruses or modified viral nucleic acids can be prepared in formulations. Such formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48. Perry's Chemical Engineer's Handbook, 4th Ed., McGraw-Hill, New York, 1963, pages 8-57 and et seq. WO 91/13546, U.S. Pat. Nos. 4,172,714, 4,144,050, 3,920,442, 5,180,587, 5,232,701, 5,208,030, GB 2,095,558, U.S. Pat. No. 3,299,566, Klingman, Weed Control as a Science, John Wiley and Sons, Inc., New York, 1961, Hance et al. Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and/or binders and/or gelling agents.

In specific embodiments, the polynucleotides, polynucleotide constructs, and expression cassettes of the disclosure can be provided to a plant using a variety of transient transformation methods known in the art. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) PNAS Sci. 91:2176-2180 and Hush et al. (1994) J. Cell Science 107:775-784, all of which are herein incorporated by reference. Alternatively, the polynucleotide can be transiently transformed into the plant using techniques known in the art. Such techniques include viral vector system and Agrobacterium tumefaciens mediated transient expression as described elsewhere herein.

The cells that have been transformed may be grown into plants in accordance with conventional ways. See, for example. McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a heterologous polynucleotide or polynucleotide construct of the invention, for example, an expression cassette of the invention, stably incorporated into their genome.

Any methods known in the art for modifying DNA in the genome of a plant can be used to modify genomic nucleotide sequences in planta, for example, to create or insert a resistance gene or even to replace or modify an endogenous resistance gene or allele thereof. Such methods include, but are not limited to, genome-editing (or gene-editing) techniques, such as, for example, methods involving targeted mutagenesis, homologous recombination, and mutation breeding. Targeted mutagenesis or similar techniques are disclosed in U.S. Pat. Nos. 5,565,350; 5,731,181; 5,756,325; 5,760,012; 5,795,972, 5,871,984, and 8,106,259; all of which are herein incorporated in their entirety by reference. Methods for gene modification or gene replacement comprising homologous recombination can involve inducing double breaks in DNA using zinc-finger nucleases (ZFN), TAL (transcription activator-like) effector nucleases (TALEN), Clustered Regularly Interspaced Short Palindromic Repeats/CRISPR-associated nuclease (CRISPR/Cas nuclease), or homing endonucleases that have been engineered endonucleases to make double-strand breaks at specific recognition sequences in the genome of a plant, other organism, or host cell. See, for example, Durai et al., (2005) Nucleic Acids Res 33:5978-90; Mani et al. (2005) Biochem Biophys Res Comm 335:447-57; U.S. Pat. Nos. 7,163,824, 7,001,768, and 6,453,242; Amould et al. (2006) J Mol Biol 355:443-58; Ashworth et al., (2006) Nature 441:656-9; Doyon et al. (2006) J Am Chem Soc 128:2477-84; Rosen et al., (2006) Nucleic Acids Res 34:4791-800; and Smith et al., (2006) Nucleic Acids Res 34:e149; U.S. Pat. App. Pub. No. 2009/0133152; and U.S. Pat. App. Pub. No. 2007/0117128; all of which are herein incorporated in their entirety by reference.

Unless stated otherwise or apparent from the context of a use, the term “gene replacement” is intended to mean the replacement of any portion of a first polynucleotide molecule or nucleic acid molecule (e.g. a chromosome) that involves homologous recombination with a second polynucleotide molecule or nucleic acid molecule using a genome-editing technique as disclosed elsewhere herein, whereby at least a part of the nucleotide sequence of the first polynucleotide molecule or nucleic acid molecule is replaced with the second polynucleotide molecule or nucleic acid molecule. It is recognized that such gene replacement can result in additions, deletions, and/or modifications in the nucleotide sequence of the first polynucleotide molecule or nucleic acid molecule and can involve the replacement of an entire gene or genes, the replacement of any part or parts of one gene, or the replacement of non-gene sequences in the first polynucleotide molecule or nucleic acid molecule.

TAL effector nucleases (TALENs) can be used to make double-strand breaks at specific recognition sequences in the genome of a plant for gene modification or gene replacement through homologous recombination. TAL effector nucleases are a class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas. 1013133107; Scholze & Boch (2010) Virulence 1:428-432; Christian et al. Genetics (2010) 186:757-761; Li el al. (2010) Nuc. Acids Res. (2010) doi: 10.1093/nar/gkq704; and Miller et al. (2011) Nature Biotechnology 29:143-148; all of which are herein incorporated by reference.

The CRISPR/Cas nuclease system can also be used to make double-strand breaks at specific recognition sequences in the genome of a plant for gene modification or gene replacement through homologous recombination. The CRISPR/Cas nuclease is an RNA-guided (simple guide RNA, sgRNA in short) DNA endonuclease system performing sequence-specific double-stranded breaks in a DNA segment homologous to the designed RNA. It is possible to design the specificity of the sequence (Cho S. W. et al., Nat. Biotechnol. 31:230-232, 2013; Cong L. et al., Science 339:819-823, 2013; Mali P. et al., Science 339:823-826, 2013; Feng Z. et al., Cell Research: 1-4, 2013).

In addition, a ZFN can be used to make double-strand breaks at specific recognition sequences in the genome of a plant for gene modification or gene replacement through homologous recombination. The Zinc Finger Nuclease (ZFN) is a fusion protein comprising the part of the FokI restriction endonuclease protein responsible for DNA cleavage and a zinc finger protein which recognizes specific, designed genomic sequences and cleaves the double-stranded DNA at those sequences, thereby producing free DNA ends (Urnov F. D. et al., Nat Rev Genet. 11:636-46, 2010; Carroll D., Genetics. 188:773-82, 2011).

Breaking DNA using site specific nucleases, such as, for example, those described herein above, can increase the rate of homologous recombination in the region of the breakage. Thus, coupling of such effectors as described above with nucleases enables the generation of targeted changes in genomes which include additions, deletions and other modifications.

The nucleic acid molecules, expression cassettes, vectors, and heterologous polynucleotides of the present disclosure may be used for transformation and/or genome editing of any plant species, including, but not limited to, monocots and dicots.

As used herein, the term “plant” includes seeds, plant cells, plant protoplasts, plant cell tissue cultures from which plants can be regenerated, plant calli, plant clumps, and plant cells that are intact in plants or parts of plants such as embryos, pollen, ovules, seeds, tubers, propagules, leaves, flowers, branches, fruits, roots, root tips, anthers, and the like. Progeny, variants, and mutants of the regenerated plants are also included within the scope of the disclosure, provided that these parts comprise the introduced polynucleotides. As used herein, “progeny” and “progeny plant” comprise any subsequent generation of a plant whether resulting from sexual reproduction and/or asexual propagation, unless it is expressly stated otherwise or is apparent from the context of usage.

As used herein, the terms “transgenic plant” and “transformed plant” are equivalent terms that refer to a “plant” as described above, wherein the plant comprises a heterologous nucleic acid molecule, heterologous polynucleotide, or heterologous polynucleotide construct that is introduced into a plant by, for example, any of the stable and transient transformation methods disclosed elsewhere herein or otherwise known in the art. Such transgenic plants and transformed plants also refer, for example, the plant into which the heterologous nucleic acid molecule, heterologous polynucleotide, or heterologous polynucleotide construct was first introduced and also any of its progeny plants that comprise the heterologous nucleic acid molecule, heterologous polynucleotide, or heterologous polynucleotide construct.

In certain embodiments, the methods involve the planting of seedlings and/or tubers and then growing such seedlings and tubers so as to produce plants derived therefrom and optionally harvesting from the plants a plant part or parts. As used herein, a “seedling” refers to a less than fully mature plant that is typically grown in greenhouse or other controlled- or semi-controlled (e.g. a cold frame) environmental conditions before planting or replanting outdoors or in a greenhouse for the production a harvestable plant part, such as, for example, a tomato fruit, a potato tuber or a tobacco leaf. As used herein, a “tuber” refers to an entire tuber or part or parts thereof, unless stated otherwise or apparent from the context of use. A preferred tuber of the present disclosure is a potato tuber.

In the methods of the disclosure involving planting a tuber, a part of tuber preferably comprises a sufficient portion of the tuber whereby the part is capable of growing into a plant under favorable conditions for the growth and development of a plant derived from the tuber. It is recognized that such favorable conditions for the growth and development of crop plants, particularly solanaceous crop plants, are generally known in the art.

In some embodiments, a plant cell is transformed with a heterologous polynucleotide encoding a protease inhibitor protein of the present disclosure. The term “expression” as used herein refers to the biosynthesis of a gene product, including the transcription and/or translation of said gene product. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide. Examples of heterologous polynucleotides and nucleic acid molecules that encode protease inhibitor proteins are described elsewhere herein.

The use of the terms “DNA” or “RNA” herein is not intended to limit the present disclosure to polynucleotide molecules comprising DNA or RNA. Those of ordinary skill in the art will recognize that the methods and compositions of the invention encompass polynucleotide molecules comprised of deoxyribonucleotides (i.e. DNA), ribonucleotides (i.e. RNA) or combinations of ribonucleotides and deoxyribonucleotides. Such deoxyribonucleotides and ribonucleotides include both naturally occurring molecules and synthetic analogues including, but not limited to, nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). The polynucleotide molecules of the disclosure also encompass all forms of polynucleotide molecules including, but not limited to, single-stranded forms, double-stranded forms, hairpins, stem-and-loop structures, and the like. Furthermore, it is understood by those of ordinary skill in the art that the nucleotide sequences disclosed herein also encompasses the complement of that exemplified nucleotide sequence.

The present disclosure is drawn to compositions and methods for enhancing the resistance of a plant to plant disease, particularly to compositions and methods for enhancing the resistance of a plant to a plant disease caused by at least one bacterial pathogen. By “disease resistance” is intended that the plants avoid the disease symptoms that are the outcome of plant-pathogen interactions. That is, pathogens are prevented from causing plant diseases and the associated disease symptoms, or alternatively, the disease symptoms caused by the pathogen is minimized or lessened.

Antimicrobial compositions and methods for controlling or preventing the growth of microbial pathogens, and in particular bacterial pathogens, on plants, plant parts and plant material are also provided herein. The active agents used to control these pathogens are protease inhibitor proteins of the present disclosure. In certain embodiments, the protease inhibitor protein has at least 80%, at least 90%, at least 95%, at least 98%, or at least 99% sequence identity to at least one of the amino acid sequences set forth in SEQ ID NOs: 1-5.

The protease inhibitor proteins, mixtures thereof or modifications thereof can be formulated into a suitable composition for use on plants, plant parts, in culturing media, in culturing facilities or nursery environments, or on plant propagation equipment. The composition can be suitably formulated to improve activity, stability and/or bio-availability and/or to limit toxicity. Formulations can contain biological salts, lipids or lipid derivatives, polysaccharides or polysaccharide derivatives, sugars or sugar derivatives, bio-friendly or approved GRAS additives.

The antimicrobial composition can be formulated in various types of formulations, such as solutions, wettable powders, soluble powders, tablets and water-soluble or dispersible granules. The antimicrobial composition can also be formulated as a concentrated stock (which is diluted in an aqueous solution prior to conventional spray application) or as a ready to use product.

A surfactant can be used as a wetting, solubilizing and penetrating agent. Suitable surfactants include anionic, cationic, non-ionic and amphoteric (e.g., zwitterionic) surfactants.

Exemplary anionic surfactants and classes of anionic surfactants suitable for use in the practice of the present disclosure include: alcohol sulfates; alcohol ether sulfates; alkylaryl ether sulfates; alkylaryl sulfonates such as alkylbenzene sulfonates and alkylnaphthalene sulfonates and salts thereof; alkyl sulfonates; mono- or di-phosphate esters of polyalkoxylated alkyl alcohols or alkylphenols; mono- or di-sulfosuccinate esters of C₁₂ to C₁₅ alkanols or polyalkoxylated C₁₂ to C₁₅ alkanols; alcohol ether carboxylates; phenolic ether carboxylates; polybasic acid esters of ethoxylated polyoxyalkylene glycols consisting of oxybutylene or the residue of tetrahydrofuran; sulfoalkylamides and salts thereof such as N-methyl-N-oleoyltaurate Na salt; polyoxyalkylene alkylphenol carboxylates; polyoxyalkylene alcohol carboxylates alkyl polyglycoside/alkenyl succinic anhydride condensation products; alkyl ester sulfates; naphthalene sulfonates; naphthalene formaldehyde condensates; alkyl sulfonamides; sulfonated aliphatic polyesters; sulfate esters of styrylphenyl alkoxylates; and sulfonate esters of styrylphenyl alkoxylates and their corresponding sodium, potassium, calcium, magnesium, zinc, ammonium, alkylammonium, diethanolammonium, or triethanolammonium salts; salts of ligninsulfonic acid such as the sodium, potassium, magnesium, calcium or ammonium salt; polyarylphenol polyalkoxyether sulfates and polyarylphenol polyalkoxyether phosphates; and sulfated alkyl phenol ethoxylates and phosphated alkyl phenol ethoxylates; sodium lauryl sulfate; sodium laureth sulfate; ammonium lauryl sulfate; ammonium laureth sulfate; sodium methyl cocoyl taurate; sodium lauroyl sarcosinate; sodium cocoyl sarcosinate; potassium coco hydrolyzed collagen; TEA (triethanolamine) lauryl sulfate; TEA (Triethanolamine) laureth sulfate; lauryl or cocoyl sarcosine; disodium oleamide sulfosuccinate; disodium laureth sulfosuccinate; disodium dioctyl sulfosuccinate; N-methyl-N-oleoyltaurate Na salt; tristyrylphenol sulphate; ethoxylated lignin sulfonate; ethoxylated nonylphenol phosphate ester; calcium alkylbenzene sulfonate; ethoxylated tridecylalcohol phosphate ester; dialkyl sulfosuccinates; perfluoro (C₆-C₁₈)alkyl phosphonic acids; perfluoro (C₆-C₁₈)alkyl-phosphinic acids; perfluoro (C₃-C₂₀)alkyl esters of carboxylic acids; alkenyl succinic acid diglucamides; alkenyl succinic acid alkoxylates; sodium dialkyl sulfosuccinates; and alkenyl succinic acid alkylpolyglykosides.

Exemplary amphoteric and cationic surfactants include alkylpolyglycosides; betaines; sulfobetaines; glycinates; alkanol amides of C₈ to C₁₈ fatty acids and Co to C₁₈ fatty amine polyalkoxylates; C₁₀ to C₁₈ alkyldimethylbenzylammonium chlorides; coconut alkyldimethylaminoacetic acids; phosphate esters of C₈ to C₁₈ fatty amine polyalkoxylates; alkylpolyglycosides (APG) obtainable from a acid-catalyzed Fischer reaction of starch or glucose syrups with fatty alcohols, in particular C₈ to C₁₈ alcohols, especially the C₈ to C₁₀ and C₁₂ to C₁₄ alkylpolyglycosides having a degree of polymerization of 1.3 to 1.6, in particular 1.4 or 1.5.

Exemplary non-ionic surfactants and classes of non-ionic surfactants include: polyarylphenol polyethoxy ethers; polyalkylphenol polyethoxy ethers; polyglycol ether derivatives of saturated fatty acids; polyglycol ether derivatives of unsaturated fatty acids; polyglycol ether derivatives of aliphatic alcohols; polyglycol ether derivatives of cycloaliphatic alcohols; fatty acid esters of polyoxyethylene sorbitan; alkoxylated vegetable oils; alkoxylated acetylenic diols; polyalkoxylated alkylphenols; fatty acid alkoxylates; sorbitan alkoxylates; sorbitol esters; C₈ to C₂₂ alkyl or alkenyl polyglycosides; polyalkoxy styrylaryl ethers; alkylamine oxides; block copolymer ethers; polyalkoxylated fatty glyceride; polyalkylene glycol ethers; linear aliphatic or aromatic polyesters; organo silicones; polyaryl phenols; sorbitol ester alkoxylates; and mono- and diesters of ethylene glycol and mixtures thereo; ethoxylated tristyrylphenol; ethoxylated fatty alcohol; ethoxylated lauryl alcohol; ethoxylated castor oil; and ethoxylated nonylphenol; alkoxylated alcohols, amines or acids, mixtures thereof as well as mixtures thereof with diluents and solid carriers, in particular clathrates thereof with urea. The alkoxylated alcohols, amines or acids are preferably based on alkoxy units having 2 carbon atoms, thus being a mixed ethoxylate, or 2 and 3 carbon atoms, thus being a mixed ethoxylate/propoxylated, and having at least 5 alkoxy moieties, suitably from 5 to 25 alkoxy moieties, preferably 5 to 20, in particular 5 to 15, in the alkoxy chain. The aliphatic moieties of the amine or acid alkoxylated may be straight chained or branched of 9 to 24, preferably 12 to 20, carbon atoms. The alcohol moiety of the alcohol alkoxylates is as a rule derived from a C₉-C₁₈ aliphatic alcohol, which may be non-branched or branched, especially monobranched. Preferred alcohols are typically 50% by weight straight-chained and 50% by weight branched alcohols.

The aforementioned surfactants may be used alone or in combination. All of these surfactant materials are well known and commercially available.

Other components of the formulation can include additional surface active agents, solvents, cosolvents, dyes, UV (ultra-violet) protectants, antioxidants, antifoams, stickers, spreaders, anti-foaming agents, preservatives, humectants, buffers, carriers, emulsifiers, wetting agents, dispersants, fixing agents, disintegrators, acid solubilizes or other components which facilitate product handling and application. These carriers, diluents, auxiliary agents and so forth are preferably selected to optimize the antibacterial action on plants or plant parts.

Solid carriers can include, for example, the following materials in fine powder or granular form: agarose/agar containing cell culture media or dried cell culture media; organic-type fertilisers; clays (e.g. kaolinite, diatomaceous earth, synthetic hydrated silicon oxide, Fubasami clay, bentonite, acid clay); talc and other inorganic minerals (e.g. sericite, quartz powder, sulfur powder, activated carbon, calcium carbonate); and chemical fertilizers (e.g. ammonium sulfate, ammonium phosphate, ammonium nitrate, ammonium chloride, urea). Liquid carriers can include, for example, cell culture media, water; alcohols (e.g. methanol, ethanol, isopropanol); ketones (e.g. acetone, methyl ethyl ketone, cyclohexanone); esters (e.g. ethyl acetate, butyl acetate); nitriles (e.g. acetonitrile, isobutyronitrile); and acid amides (e.g. dimethylformamide, dimethylacetamide), as well as dilute bases (e.g. sodium hydroxide, potassium hydroxide and amines)

Other auxiliary agents can include, for example, adhesive agents and dispersing agents, such as casein, gelatin, polysaccharides (e.g. powdered starch, gum arabic, cellulose derivatives, alginic acid, chitin), lignin derivatives and synthetic water-soluble polymers (e.g. polyvinyl alcohol, polyvinyl pyrrolidone, polyacrylic acid); salts (eg. citrate, chloride, sulphate, acetate, ammonium, bicarbonate, phosphate salts and like) and stabilizers such as PAP (isopropyl acid phosphate), BHT (2,6-di-tert-butyl-4-methylphenol), BHA (2-/3-tert-butyl-4-methoxyphenol), vegetable oils, mineral oils, phospholipids, waxes, fatty acids and fatty acid esters.

Conventional plant growth regulators, herbicides, fungicides, bactericides, insecticides, nematicides, acaricides, biochemical pesticides, plant produced pesticides (botanicals), cell culture media components or plant nutrients and so forth can also be incorporated into the antimicrobial compositions of the present disclosure.

The antimicrobial composition may be diluted in water, water organic mixture or with liquid carrier and sprayed or applied in controlled environments on the plant or plant material to be treated or used to wash plant materials or environment/systems/equipment or mixed with cell culture media or plant propagation media. Alternatively, the composition may be directly applied to the soil (in which the plant will be grown or is growing).

### EMBODIMENTS

### The Following Numbered Embodiments Also Form Part of the Present Disclosure:

1. A plant, or a plant cell thereof, with enhanced resistance to at least one bacterial pathogen, the plant comprising a heterologous polynucleotide encoding a protease inhibitor protein having at least 80%, at least 90%, at least 95%, at least 98%, or at least 99% sequence identity to at least one of the amino acid sequences set forth in SEQ ID NOs: 1-5.
2. The plant of embodiment 1, wherein the polynucleotide encoding the protease inhibitor protein has at least 80%, at least 90%, at least 95%, at least 98%, or at least 99% sequence identity to at least one of the nucleotide sequences set forth in SEQ ID NOs: 6-15.
3. The plant of embodiment 1 or embodiment 2, wherein the polynucleotide is operably linked to a promoter functional in a plant cell.
4. The plant of embodiment 3, wherein the promoter is a pathogen-inducible promoter, a constitutive promoter, a tissue-preferred promoter, a wound-inducible promoter, or a chemical-regulated promoter.
5. The plant of any one of embodiments 1-4, wherein the at least one bacterial pathogen is of the order Enterobacterales, optionally wherein the at least one bacterial pathogen is of the family Pectobacteriaceae.
6. The plant of any one of embodiments 1-5, wherein the at least one bacterial pathogen is a Pectobacterium spp.
7. The plant of any one of embodiments 1-6, wherein the at least one bacterial pathogen causes a bacterial soft rot.
8. The plant of any one of embodiments 1-7, wherein the plant is a solanaceous plant.
9. The plant of embodiment 8, wherein the solanaceous plant is a potato plant.
10. The plant of any one of embodiments 1-9, wherein the plant is Solanum chocoense.
11. A method of enhancing the resistance of a plant to at least one bacterial pathogen, the method comprising: modifying at least one plant cell to comprise a polynucleotide encoding a protease inhibitor protein having at least 80%, at least 90%, at least 95%, at least 98%, or at least 99% sequence identity to at least one of the amino acid sequences set forth in SEQ ID NOs: 1-5.
12. The method of embodiment 11, wherein the polynucleotide is stably incorporated into the genome of the plant cell.
13. The method of embodiment 11 or embodiment 12, wherein the plant cell is regenerated into a plant comprising in its genome the polynucleotide.
14. The method of any one of embodiments 11-13, wherein modifying at least one plant cell to comprise the polynucleotide comprises introducing a heterologous polynucleotide encoding the protease inhibitor protein into at least one plant cell.
15. The method of any one of embodiments 11-14, wherein the polynucleotide is operably linked to a promoter functional in a plant cell.
16. The method of embodiment 15, wherein the promoter is a pathogen-inducible promoter, a constitutive promoter, a tissue-preferred promoter, a wound-inducible promoter, or a chemical-regulated promoter.
17. The method of any one of embodiments 11-13, wherein modifying at least one plant cell to comprise a polynucleotide comprises using genome editing to modify the nucleotide sequences of a native or non-native gene in the genome of the plant cell to comprise the polynucleotide encoding the protease inhibitor protein.
18. The method of any one of embodiments 11-17, wherein the polynucleotide encoding the protease inhibitor protein has at least 80%, at least 90%, at least 95%, at least 98%, or at least 99% sequence identity to at least one of the nucleotide sequences set forth in SEQ ID NOs: 6-15.
19. The method of any one of embodiments 11-18, further comprising selecting for a plant or a plant cell having enhanced resistance to at least one bacterial pathogen as compared to a corresponding control plant or plant cell without the polynucleotide.
20. The method of any one of embodiments 11-19, wherein the at least one bacterial pathogen is of the order Enterobacterales, optionally wherein the at least one bacterial pathogen is of the family Pectobacteriaceae.
21. The method of any one of embodiments 11-20, wherein the at least one bacterial pathogen is a Pectobacterium spp.
22. The method of any one of embodiments 11-21, wherein the at least one bacterial pathogen causes a bacterial soft rot.
23. The method of any one of embodiments 11-22, wherein the plant is a solanaceous plant.
24. The method of embodiment 23, wherein the solanaceous plant is a potato plant.
25. A plant produced by the method of any one of embodiments 11-24. 26. A fruit, tuber, leaf, or seed of the plant of any one of embodiments 1-10 and 25, wherein the fruit, tuber, leaf, or seed comprises the heterologous polynucleotide.
27. A nucleic acid molecule comprising a nucleotide sequence selected from the group of: (a) the nucleotide sequence set forth in SEQ ID NO: 6, 7, 8, 9, 10, 11, 12, 13, 14, or 15; (b) a nucleotide sequence encoding the amino acid sequence set forth in SEQ ID NO: 1, 2, 3, 4, or 5; (c) a nucleotide sequence having at least 80%, at least 90%, at least 95%, at least 98%, or at least 99% sequence identity to at least one of the nucleotide sequences set forth in SEQ ID NOs: 6, 7, 8, 9, 10, 11, 12, 13, 14, and 15; and (d) a nucleotide sequence encoding an amino acid sequence having at least 80%, at least 90%, at least 95%, at least 98%, or at least 99% sequence identity to at least one of the amino acid sequences set forth in SEQ ID NOs: 1, 2, 3, 4, and 5.
28. The nucleic acid molecule of embodiment 27, wherein the nucleic acid molecule is capable of conferring resistance to a plant disease caused by at least one bacterial pathogen to a plant comprising the nucleic acid molecule.
29. The nucleic acid molecule of embodiment 27 or embodiment 28, wherein the nucleotide sequence is not naturally occurring.
30. The nucleic acid molecule of any one of embodiments 27-29, wherein the nucleic acid molecule is an isolated nucleic acid molecule.
31. An expression cassette comprising the nucleic acid molecule of any one of embodiments 27-30 and an operably linked heterologous promoter.
32. A vector comprising the nucleic acid molecule of any one of embodiments 27-30 or the expression cassette of embodiment 31.
33. A host cell comprising the nucleic acid molecule of any one of embodiments 27-30 or the expression cassette of embodiment 31 or the vector of embodiment 32.
34. The host cell of embodiment 33, wherein the host cell is a plant cell, a bacterium, a fungal cell, or an animal cell.
35. The host cell of embodiment 33 or embodiment 34, wherein the host cell is a solanaceous plant cell.
36. The host cell of embodiment 35, wherein the solanaceous plant cell is a potato plant cell.
37. A method of limiting a plant disease caused by at least one bacterial pathogen in agricultural crop production, the method comprising: planting a seedling, tuber, or seed of the plant of any one of embodiments 1-10; and growing the seedling, tuber, or seed under conditions favorable for the growth and development of a plant resulting therefrom.
38. The method of embodiment 37, further comprising harvesting at least one fruit, tuber, leaf and/or seed from the plant.
39. A method for identifying a plant that displays newly conferred or enhanced resistance to a plant disease caused by at least one bacterial pathogen, the method comprising: detecting in the plant, or in at least one part or cell thereof, the presence of a protease inhibitor nucleotide sequence having at least 80%, at least 90%, at least 95%, at least 98%, or at least 99% sequence identity to at least one of the nucleotide sequences set forth in SEQ ID NOs: 6-15.
40. The method of embodiment 39, wherein the plant disease is a bacterial soft rot or blackleg.
41. The method of embodiment 39 or embodiment 40, wherein the plant is a solanaceous plant.
42. The method of embodiment 41, wherein the solanaceous plant is a potato plant.
43. The method of any one of embodiments 39-42, wherein the presence of the protease inhibitor nucleotide sequence is detected by detecting at least one marker within the protease inhibitor nucleotide sequence.
44. The method of any one of embodiments 39-43, wherein detecting the presence of the protease inhibitor nucleotide sequence comprises PCR amplification, nucleic acid sequencing, nucleic acid hybridization, or an immunological assay for the detection of the protease inhibitor protein encoded by the protease inhibitor nucleotide sequence.
45. A plant identified by the method of any one of embodiments 39-44.
46. A fruit, tuber, leaf, or seed of the plant of embodiment 45.
47. A method for introducing at least one protease inhibitor gene into a plant, the method comprising: (a) crossing a first plant comprising in its genome at least one copy of at least one protease inhibitor gene with a second plant lacking in its genome the at least one protease inhibitor gene, whereby at least one progeny plant is produced; and (b) selecting at least one progeny plant comprising in its genome the at least one protease inhibitor gene.
48. The method of embodiment 47, wherein the first plant is a Solanum chocoense plant and the second plant is not a Solanum chocoense plant.
49. The method of embodiment 47 or embodiment 48, wherein the second plant is a Solanum tuberosum plant.
50. The method of any one of embodiments 47-49, wherein at least one protease inhibitor gene comprises a nucleotide sequence selected from the group of: (a) the nucleotide sequence set forth in SEQ ID NO: 6, 7, 8, 9, 10, 11, 12, 13, 14, or 15; (b) a nucleotide sequence encoding the amino acid sequence set forth in SEQ ID NO: 1, 2, 3, 4, or 5; (c) a nucleotide sequence having at least 80%, at least 90%, at least 95%, at least 98%, or at least 99% sequence identity to at least one of the nucleotide sequences set forth in SEQ ID NOs: 6, 7, 8, 9, 10, 11, 12, 13, 14, and 15; and (d) a nucleotide sequence encoding an amino acid sequence having at least 80%, at least 90%, at least 95%, at least 98%, or at least 99% sequence identity to at least one of the amino acid sequences set forth in SEQ ID NOs: 1, 2, 3, 4, and 5.
51. The method of any one of embodiments 47-50, further comprising (i) backcrossing at least one selected progeny plant of (b) to a plant that is of the same species and genotype as second plant or of the same species as the second plant and lacking in its genome the at least one protease inhibitor gene, whereby at least one progeny plant is produced from the backcrossing; and (ii) selecting at least one progeny plant comprising in its genome the at least one protease inhibitor gene that is produced from the backcrossing of (i).
52. A progeny plant according to any one of embodiments 47-51.
53. The progeny plant of embodiment 52, wherein the progeny plant is not Solanum chocoense.
54. A fruit, tuber, leaf, or seed of the plant of embodiment 52 or 53.
55. Use of the plant, fruit, tuber, leaf, or seed of any one of embodiments 1-10, 25, 26, 45, 46, and 52-54 in agriculture.
56. A human or animal food product comprising, or produced using, the plant, fruit, tuber, leaf and/or seed of any one of embodiments 1-10, 25, 26, 45, 46, and 52-54.
57. A protease inhibitor polypeptide comprising an amino acid sequence selected from the group: (a) the amino acid sequence set forth in SEQ ID NO: 1, 2, 3, 4, or 5; (b) the amino acid sequence encoded by the nucleotide sequence set forth in SEQ ID NO: 6, 7, 8, 9, 10, 11, 12, 13, 14, or 15; and (c) an amino acid sequence having at least 80%, at least 90%, at least 95%, at least 98%, or at least 99% sequence identity sequence identity to at least one of the amino acid sequences set forth in SEQ ID NOs: 1, 2, 3, 4, and 5.
58. The protease inhibitor polypeptide of embodiment 57, wherein the polypeptide is capable of conferring resistance to a plant disease caused by at least one bacterial pathogen to a plant comprising the polypeptide.
59. An antimicrobial composition comprising: at least one protease inhibitor protein having at least 80%, at least 90%, at least 95%, at least 98%, or at least 99% sequence identity to at least one of the amino acid sequences set forth in SEQ ID NOs: 1-5.
60. The antimicrobial composition of embodiment 59, wherein the composition comprises two, three, four, or five protease inhibitor proteins having an amino acid sequence selected from SEQ ID NOs: 1-5.
61. The antimicrobial composition of embodiment 59 or embodiment 60, further comprising a carrier.
62. The antimicrobial composition of any one of embodiments 59-61, further comprising one or more of a filler, a diluent, a dye, an adjuvant, an emulsifier, a dispersing agent, a wetting agent, a thickener, a thixotropic agent, or a defoaming agent.
63. The antimicrobial composition any one of embodiments 59-62, wherein the composition is capable of treating or preventing a bacterial soft rot on a plant or a plant part.
64. A method of preventing or controlling microbial growth on a plant or a plant part, the method comprising: contacting the surface of the plant or plant part with the antimicrobial composition of any one of embodiments 59-63.
65. The method of embodiment 64, wherein the plant or plant part is dipped in the antimicrobial composition.
66. The method of embodiment 64, wherein the plant or plant part is sprayed or coated with the antimicrobial composition.
67. The method of any one of embodiments 64-66, wherein the plant part is a harvested plant part.
68. The method of any one of embodiments 64-66, wherein the plant part is a fruit, tuber, leaf, or seed.
69. The method of any one of embodiments 64-68, wherein the plant or plant part is a potato plant or potato plant part.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

The following examples are offered by way of illustration and not by way of limitation.

### EXAMPLES

### Example 1: M6 Protein Extracts Reduced Bacterial Exoenzyme Activity but were not Bactericidal

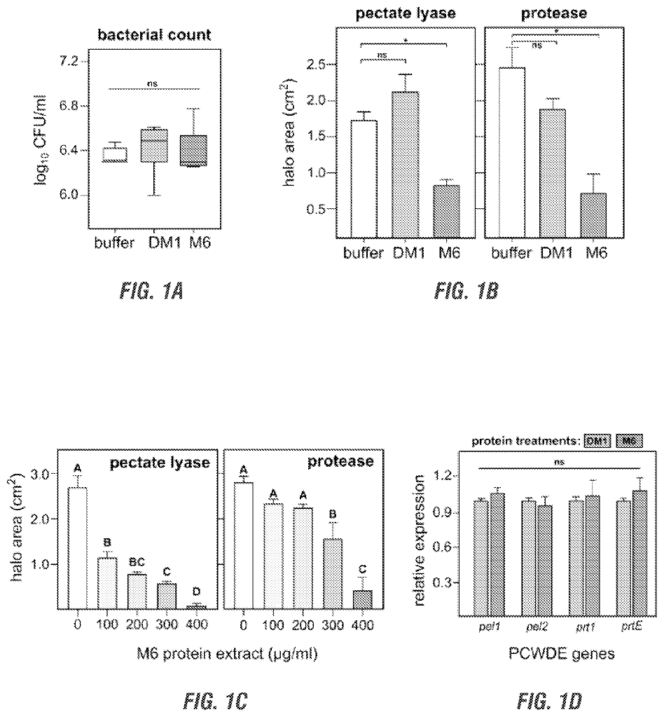

Bactericidal effects of S. chacoense line M6 (resistant) were initially evaluated by measuring bacterial (P. brasiliense Pb1692) multiplication in the presence of crude tuber protein extracts in vitro and were compared to extracts from the susceptible potato line DM1 (S. tuberosum) Neither the protein extract of M6 or DM1 affected bacterial multiplication in vitro (FIG. 1A, ANOVA p=0.69). Next, effects of protein extracts on bacterial virulence were evaluated with in vitro assays that measure plant cell wall degrading enzyme (PCWDE) activity in the supernatant. Incubation with M6 protein extracts reduced pectate lyase and protease activity (FIG. 1B, ANOVA Dunnett's post hoc p<0.05), whereas DM1 protein did not reduce pectate lyase and protease activity (p=0.15). A dose-dependent response was also performed, and the results show that increased protein concentration was associated with decreased PCWDE activity (FIG. 1C, ANOVA p<0.05). Importantly this assay does not differentiate between an effect on activity (e.g., antagonistic binding) or synthesis of those enzymes via gene expression changes. Therefore, plant protein effects on bacterial gene expression were measured. Neither DM1 nor M6 protein extracts had any effect on gene expression of the tested pel and prt genes (FIG. 1D).

### Example 2: Incubation with M6 and DM1 Protein Extracts Caused Differential Protein Expression Profiles in Pectobacterium and Indicated Broader Effects on Inhibiting Bacterial Virulence

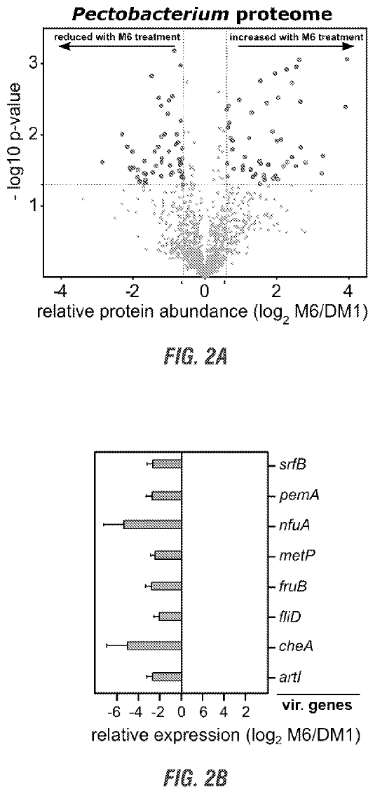

To further characterize effects of the M6 protein extract on virulence, the extracellular and intercellular bacterial proteomes were analyzed using non-targeted proteomics approach. We identified 1158 bacterial proteins/peptides, of which 131 were differentially expressed per plant variety (89 intracellular, 42 extracellular, FIG. 2A; FC>1.5 to <−1.5, p<0.05). The analysis compared effects of the protein extract from the susceptible DM1 to the resistant M6. Pectobacterium that were incubated with M6 protein extracts had reduced abundances of chemotaxis and flagellar protein, peptidase, metalloprotease, and some other virulence proteins. Further, proteins involved in stress, ATP-binding cassette (ABC) transporter systems, and ribosomal proteins were upregulated with M6 protein extracts. To validate the proteomic data, some virulence genes were tested for their expression using qRT-PCR after growing them in M6 and DM1 protein extracts. Expression studies validated the proteomic data by showing lower abundance of the selected virulence genes when bacteria were grown in M6 extract compared to DM1 (FIG. 2B).

### Example 3: M6 Encodes More Protease Inhibitor Genes than DM1

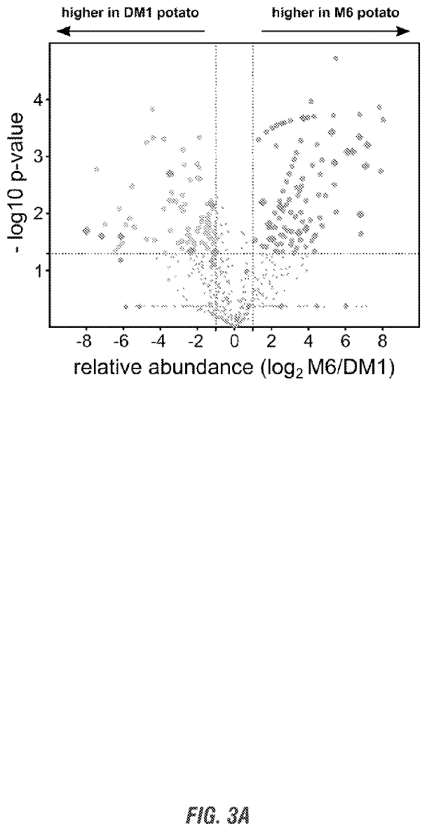

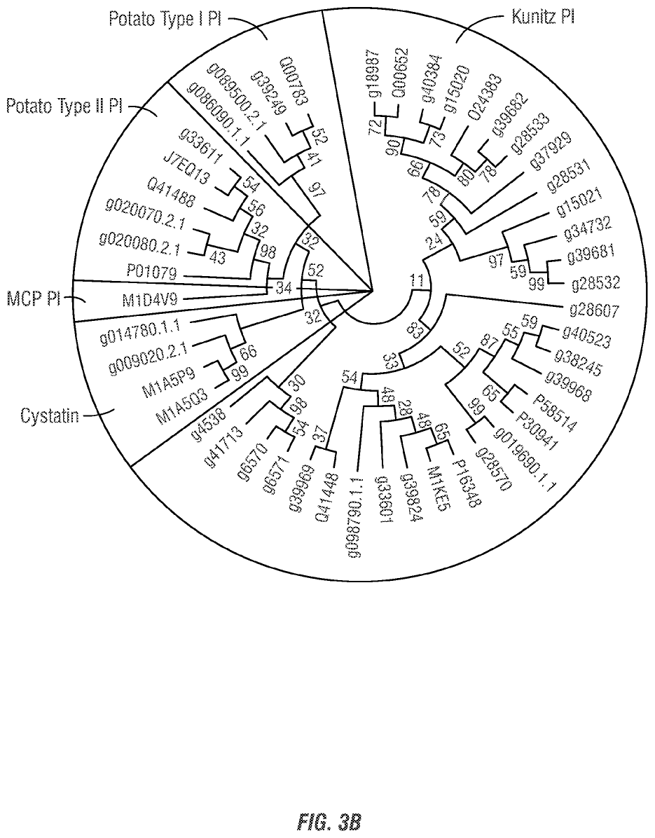

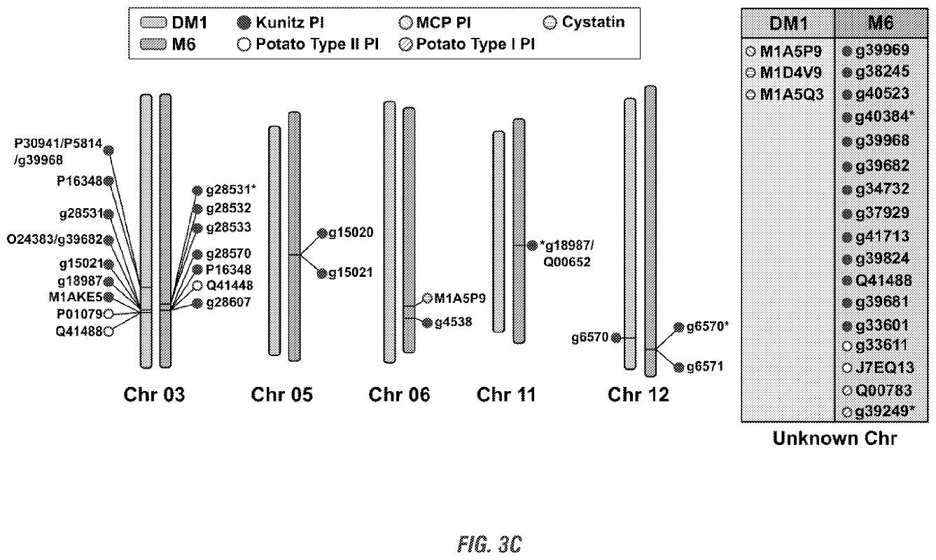

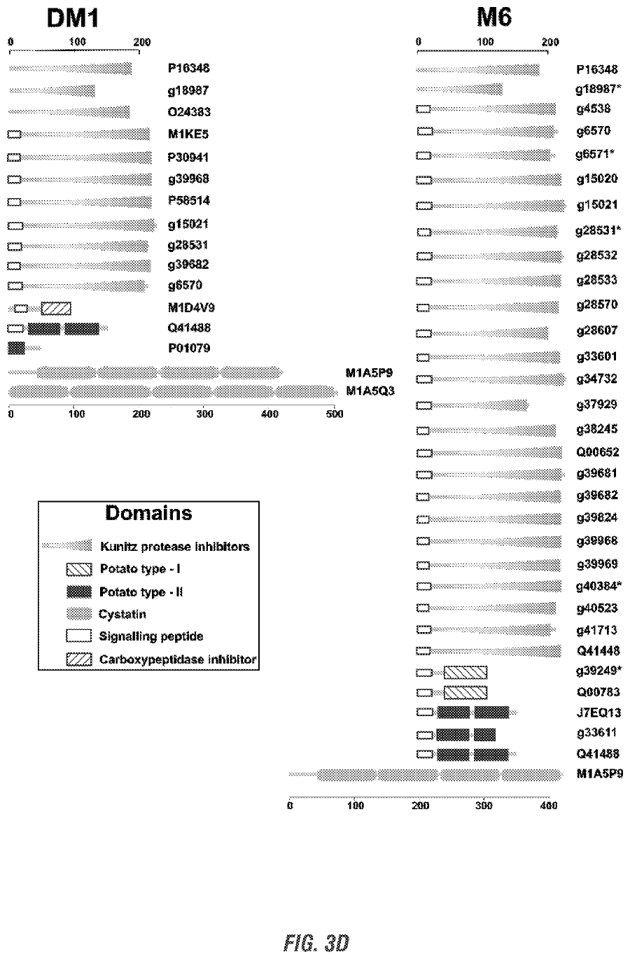

LC-MS proteomics was performed on the DM1 and M6 tubers and identified 778 protein/peptides were present in extracts. This can be considered a low number of proteins, but this was expected due to the extraction and cleaning process (membrane dialysis with a 6-8 kD cutoff, and less soluble proteins were precipitated and removed using 20% ammonium sulphate). Also, trypsin digestion is a component of the proteomics method, and the extracts included potato-encoded trypsin inhibitors. The data indicated protease inhibitors (PIs) were a major differential factor in the protein extracts (FIG. 3A). A total of 48 PIs were identified, and M6 protein extracts contained more PIs (32) relative to DM1 (16). These 48 expressed PIs were out of a potential 127 and 61 PI genes found in the M6 and DM1 genomes, respectively. Next, domain (PI type) and sequence-based phylogenetic analysis was performed on the 48 expressed PIs. Five classes of protease inhibitors PIs were observed, with Kunitz-trypsin PI being the most abundant (FIG. 3B, FIG. 3D). The proteins were also mapped to genetic loci and indicate diversity in PI genetics between potato species. In DM1, all but one PI mapped to a single locus on chromosome 3 (FIG. 3C). In contrast, M6 had more PI genes, and these genes mapped to at least five different chromosomes.

### Example 3: The M6 Protein Extract Inhibited Bacterial Protease Activity, Motility, and Affected Cell Morphology

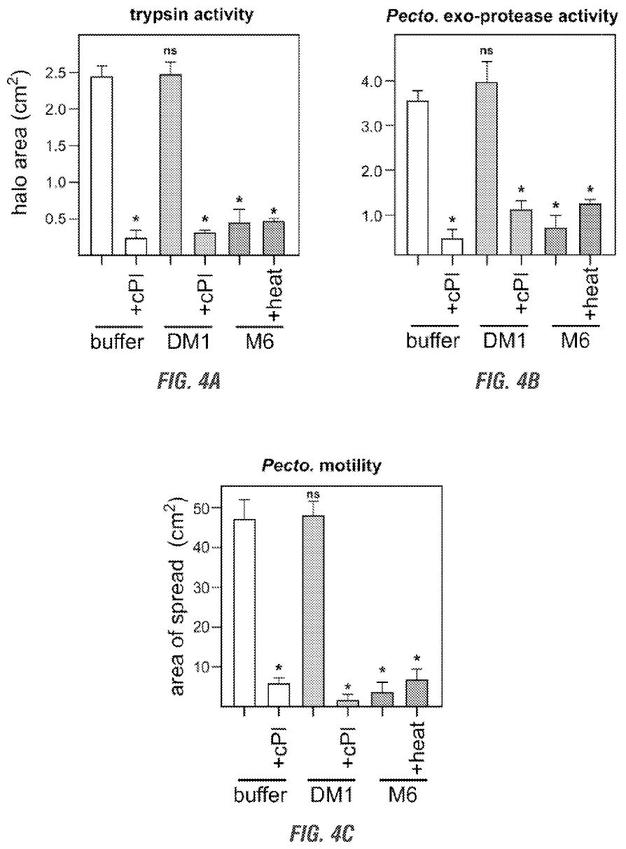

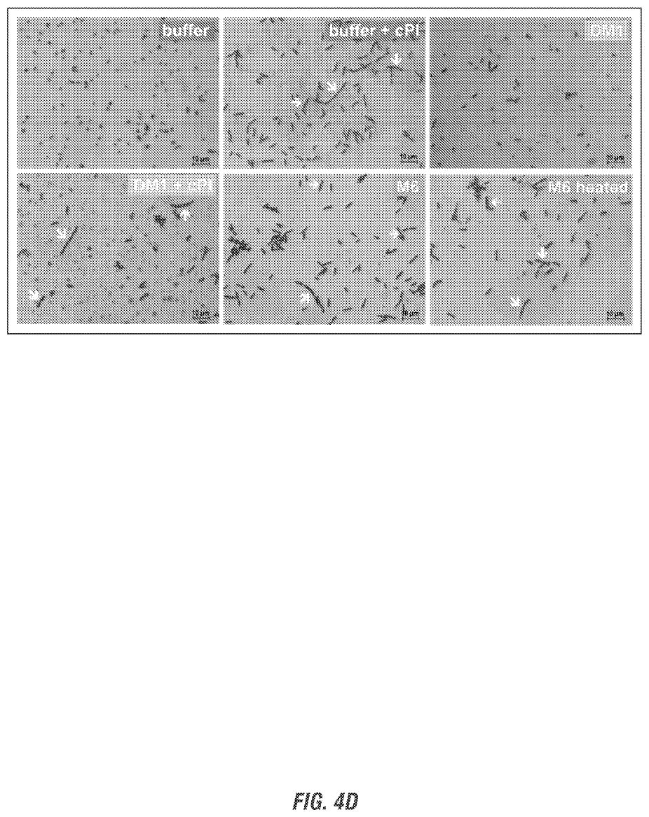

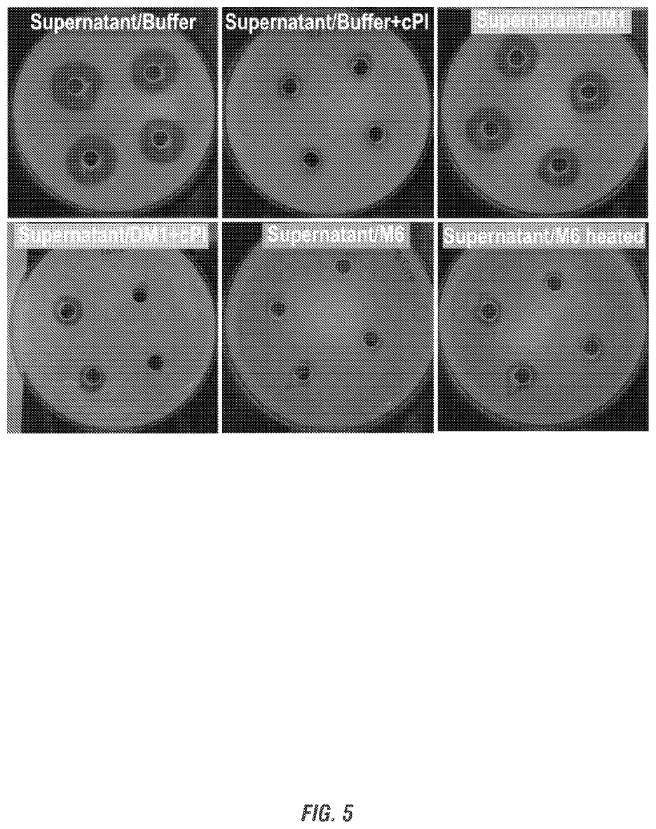

As the proteomics and genomics data indicated PIs as a major difference between resistant and susceptible potato, and PIs are known to affect virulence of several bacteria, the protein extracts were further evaluated by comparing effects on Pectobacterium compared to authentic standards as controls (protease inhibitor cocktail, cPI). PIs are also heat stable, and so heating can reduce the complexity of the extract to emphasize PIs. Trypsin inhibition activity was observed for M6 protein extracts and this activity was maintained after heating (FIG. 4A). This activity was consistent with Pectobacterium cultures, as regular and heat-treated M6 extracts inhibited exo-proteases, similar to the cPI positive control, and this was not observed in DM1 (FIG. 4B, FIG. 5).

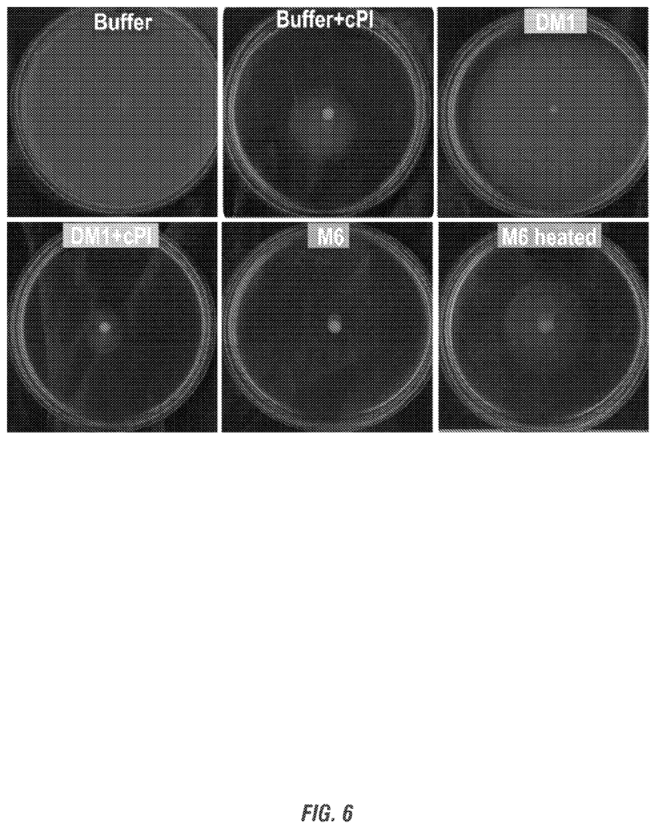

The M6 protein extract was then tested for effects on two key virulence factors: motility and cell morphology. M6 extracts (regular and heated) inhibited Pectobacterium motility and was consistent with effects of the cPI control (FIG. 4C, FIG. 6). Pectobacterium cell morphology shifts in response to the environment and this has consequences on virulence. Under favorable conditions, Pectobacterium is rod shaped and approximately 2 μm long. Pectobacterium incubated with M6 protein transitioned to a filamentous shape (>5 μm) and this was consistent with the cPI control, but cells remained in a normal shape when incubated with DM1 (FIG. 4D). Importantly, the DM1 protein extract did not exhibit inhibitory activity on any of the virulence phenotypes, nor did it inhibit trypsin, although all effects of M6 could be mimicked by adding cPI to the DM1 protein extracts.

### Example 4: Cloned and Purified Protease Inhibitors Reduced Pectobacterium Motility, Protease Activity, and Disease Symptoms

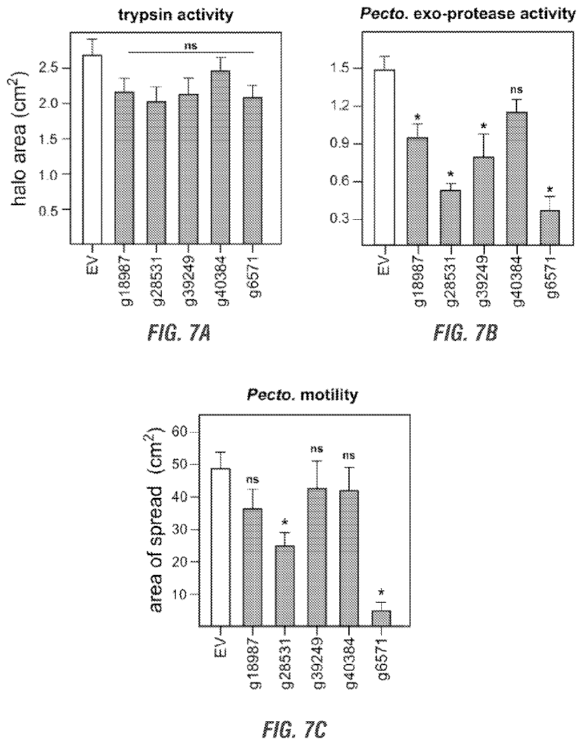

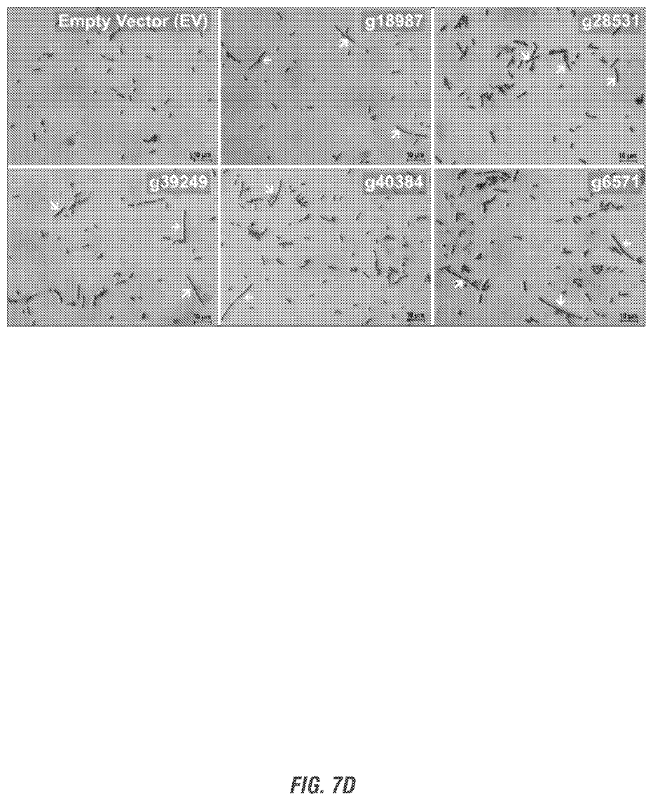

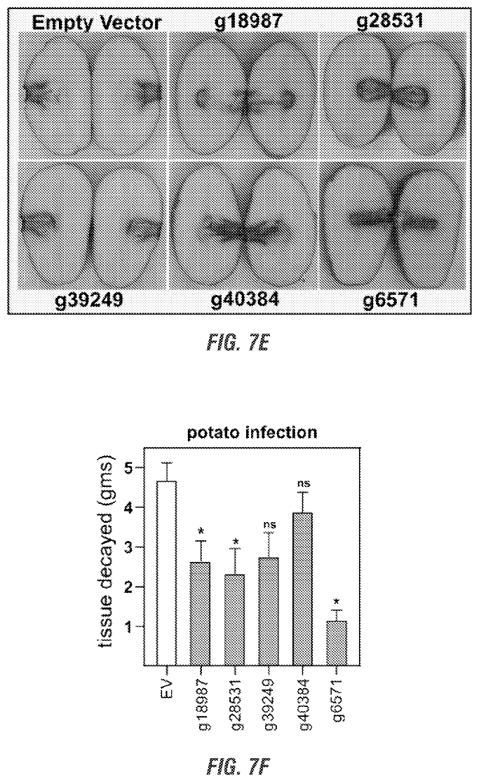

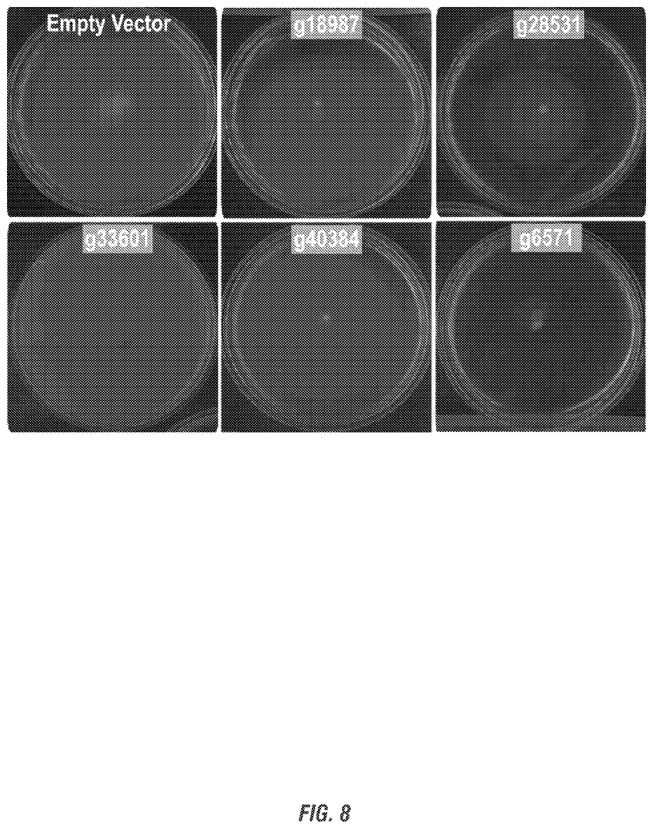

Five of the 48 expressed M6 PIs were cloned into an expression vector and electroporated into E. coli BL21, and the transformants were used for protein expression (g18987, g28531, g39249, g40384, and g6571). Each M6 PI protein was isolated using columns and dialysis and tested for effects on exo-protease activity, motility, and cell morphology. In contrast to the crude protein extracts, none of the five purified PIs inhibited trypsin (although, all PIs trended towards inhibition)(FIG. 7A). This is likely because although the cloned PIs were within the Kunitz Type family, none contained the conserved residues required for trypsin inhibition. Four of the five cloned and purified proteins inhibited Pectoberium exo-protease activity (FIG. 7B). Two of these five PIs inhibited Pectobacterium motility (g28531 and g6571), and all five PIs induced the filamentous morphological phenotype (FIG. 7C-D, FIG. 8). In addition to effects on virulence factors, we also tested if the cloned M6 PIs altered disease severity. In this assay, tubers from a commercial potato (S. tuberosum) were co-inoculated with Pectobacterium cells and each of the five cloned M6 PIs. Three of the cloned M6 PIs reduced disease severity as measured by tissue decay (g18987, g28531, and g6571, FIG. 7E-F), supporting an association between virulence inhibition and disease resistance.

### Discussion for Examples 1-4

Plants have evolved a diverse set of PIs that both regulate plant protease activity and facilitate defense against pests and pathogens. Our findings demonstrate that protease inhibitors from S. chacoense M6 contribute to plant resistance to Pectobacterium. Our study observed clear effects of S. chacoense tuber proteins for disease resistance. We cloned PI genes, purified proteins, and tested the individual PIs to identify new Pectobacterium resistance gene. Both crude tuber protein extracts containing many PIs and individual PIs affected Pectobacterium virulence including PCWDE activity, swimming motility, and induced filamentous cell formation. The S. chacoense M6 protein extracts did not alter gene expression of some major bacterial exo-proteases or pectate lyases, supporting that the extracts are directly inhibiting the enzymes rather than enzyme synthesis. Interestingly, the S. chacoense M6 protein extract did reduce the abundance of several other Pectobacterium virulence proteins. This inhibition was validated by measuring expression of a subset of virulence genes including pectin methyl esterase, flagellar, and chemotaxis proteins. This supports that the action of the S. chacoense M6 PIs are two-fold: i) direct inhibition of extracellular protease activity and ii) indirect effects on virulence pathways.

The mechanism by which protease inhibitors affect swimming motility and cell shape is unknown, but these phenotypes are closely related. The large number of bacterial proteins that are differentially expressed when exposed to the S. chacoense M6 protein extract supports that PIs have global effects on Pectobacterium morphology and metabolism. These morphological effects may be similar to the non-virulent, filamentous cell morphology found in Dickeya cells and the reduced exo-enzyme, motility, and biofilm formation when the flagellar sigma factor FliA is deleted.

The proteomic and genomic analyses show that S. chacoense M6 encodes and expresses more PIs than the susceptible S. tuberosum DM1. Further, S. chacoense M6 has more PI loci across more chromosomes and more diversity in PI type. The reduced diversity in PI genetics may be an effect of domestication and breeding, and perhaps indicates a loss of PI diversity in modern cultivars that contributes to disease susceptibility. In potato, this phenomenon occurred with the glycoalkaloids, a group of protective chemicals that are exceptionally high in wild species, but that have been bred to be low in modern cultivars. Further, the diverse set of protease inhibitors in S. chacoense M6 may act synergistically and with other resistance processes, for example with S. chacoense M6 metabolites that inhibit quorum sensing. Such a diverse set of genes and resistance traits is expected given the multi-genic nature of potato resistance.

Despite numerous reports of wild potato with resistance to Pectobacterium, until now, no candidate resistance genes from potato were described. Unlike metabolite-based resistance or plant cell wall fortification, this PI-based resistance may be conferred by individual genes, making it an attractive option to develop new cultivars with increased resistance to Pectobacterium. As a proof of concept for this, we selected five protease inhibitors that were in high abundance in S. chacoense M6 protein extracts or that are present in M6 but not DM1. The purified PI proteins (except for g40384) significantly reduced Pectobacterium exo-protease activity, perhaps by direct inhibition of active sites or by inducing conformational changes to the exo-protease structure. The PIs induce Pectobacterium cell elongation, which has been reported to be a non-virulent state described in the closely related pathogen Dickeya. Two of the protease inhibitors, g28531 and g6571, also reduced bacterial swimming motility, which further supports that PIs induce this distinct non-virulent bacterial state. To clarify the effect of PIs in Pectobacterium virulence, tuber infection assays showed that some of the PIs significantly reduced disease severity. This supports the potential for PIs to be used in breeding programs and as purified proteins in the food and agriculture industry to manage necrotrophic bacterial pathogens.

### Methods for Examples 1-4

### Plant Materials, Bacterial Strains, and Chemicals

Solanum tuberosum DM1 (sometimes known as DM1-3) and S. chacoense M6 were grown in a greenhouse at 18-24° C. with 16 h day length. Plants were gown in ProMix BX general purpose mix and fertilized with Osmocote Plus 15-9-12 (Scotts-MiracleGro, OH, USA). Tubers and 1-month old stems were used for protein extraction. NaCl, ethylenediaminetetraacetic acid (EDTA), thiourea, dithiothreitol (DTT), phenylmethylsulfonyl fluoride (PMSF), Tris-HCl, ammonium sulphate, polyvinylpolypyrrolidone (PVP), and hydrochloric acid were purchased from Fisher Chemicals (Thermo Fisher Scientific, MA, USA). Pectobacterium brasiliense strain 1692 (Pb1692) was used for all experiments in this study. Nutrient broth (NB), agar, gelatin, skim milk powder, trypsin, and protease inhibitor cocktail (cPI) were purchased from Difco Laboratories (Thermo Fisher Scientific, MA, USA).

### Crude Potato Protein Extraction and Quantitation

Protein was extracted from M6 and DM1 potato tubers. The tubers were ground to a powder with a mortar and pestle in liquid nitrogen and 30 ml of protein extraction buffer was added in a centrifuge tube at 1:3 (v:v)(250 mM NaCl, 10 mM EDTA, 10 mM thiourea, and 1% PVP, suspended in 20 mM Tris-HCl at pH 7.0). The mixture was then vortexed for 30 min at 4° C., followed by centrifugation (10,000 g; 30 min; 4° C.). Twenty-five microliters of the supernatant were collected, ammonium sulfate was added to a final concentration of 20%, and the solution was incubated at 4° C. for 30 minutes. The mixture was then centrifuged (11,000 g; 30 min; 4° C.) and the supernatant was collected and dialyzed overnight at 4° C. with Tris buffer (20 mM tris-HCl, pH 7.0) in dialysis membrane (regenerated cellulose membrane: 6-8 kD cutoff, Spectrum Labs). For some experiments, dialyzed protein extracts were heated at 70° C. for 20 min and briefly centrifuged to collect the supernatant. The protein extracts were quantified using bicinchoninic (BCA) assay following the manufacturer's instructions (ThermoFisher Scientific, MA, USA). Absorbance was measured at 550 nm on a plate reader and total protein concentrations were calculated based on a bovine serum albumin standard curve.

### Bacterial Multiplication, Exo-Enzyme, and Motility Assays

Bacterial cultures were grown overnight in NB at 28° C. with shaking at 220 rpm. The overnight culture was centrifuged and resuspended into sterile water, which was used as a source of inoculum for studying bacterial responses to protein extracts. Bacteria (10 ul of 10⁸ CFU·ml⁻¹ stock) were inoculated into NB (100× bacterial culture volume, 1 ml) plus protein extract (400 μg·ml⁻¹ stock concentration) or into cPI and NB as positive control or into buffer (20 mM tris-HCl, pH 7.0) and NB as control. Bacteria were then incubated for 15 h at 28° C. with shaking at 220 rpm. Multiplication was measured by making serial dilutions and plating onto NB agar plates. Results were recorded as CFU·ml⁻¹.

For exo-enzyme assays, the culture tubes were centrifuged (8,000 g for 5 min). The bacterial pellets were used for RNA extraction and the supernatant was filter sterilized to evaluate its exo-enzyme activity in pectate lyase (Pel) or protease (Prt) assays. Pectate lyase and protease activity assays were performed in plates as previously described. Protease and trypsin activity assays were performed in plates containing 1% dry skim milk and 0.8% agar. Cores of 5 mm diameter were extracted from the plates with a sterile core borer and each well was filled with 50 μl sterile bacterial supernatant. The plates were then incubated for 18 h (for Pel assays) and 48 h (for Prt assays) at room temperature. Pel plates were washed with 4N HCl to visualize the halos. No treatment was needed for halo visualization in Prt plates. The plates were digitally scanned and activity of the enzymes were expressed as the area of the observed halos measured using ImageJ 1.52v (Wayne Rasband, NIH-USA).

Bacterial swimming motility was determined using semisolid tryptone medium (1% tryptone, 3% NaCl, 0.3% agar). The protein extract-bacterial mixture (concentrations as mentioned above) was incubated for 2 h, and 2 μl of the mixture was dropped into the center of motility plates and incubated at 28° C. for 15 h. The plates were digitally scanned and the circular turbid zones were measured using ImageJ (Wayne Rasband, NIH-USA).

RNA Extraction, cDNA Preparation, and qRT-PCR

The Pb1692 cell pellets (after culturing bacteria in protein extract, as described above) were used for RNA extraction using TRIzol reagent following the user's guide (ThermoFisher Scientific, MA, USA). A total of 1 μg of extracted RNA was reverse transcribed using an iScript™ cDNA synthesis kit (Bio-Rad Laboratories Inc, CA, USA) to obtain cDNA. The quantitation of transcripts: pel1, pel2 (pectate lyase); prt1, prtE (protease); and additional genes were performed by qRT-PCR. Additional primer sequences for gene expression by qRT-PCR are listed in Table 2.

TABLE 2
Primers used for bacterial gene expression and cloning of potato genes.

| | | |
|---|---|---|
| Genes | Forward 5′→3′ | Reverse 5′→3′ |

Primers used for qRT-PCR

| | | |
|---|---|---|
| srfB-virulence factor | AGCGACGAACTGGGTGAATT | CCCTCTGAGCGCCACTTTTA |
| | (SEQ ID NO: 16) | (SEQ ID NO: 17) |
| pemA-pectin esterase A | GGACGCAGCTTCTTCTCACA | GGCTGATCTTTCACGTCGGT |
| | (SEQ ID NO: 18) | (SEQ ID NO: 19) |
| nfuA-Fe/S biogenesis | GCTGATGGAACGTGTGGAGT | GCCAAATTGCAGAATCGCCA |
| protein | (SEQ ID NO: 20) | (SEQ ID NO: 21) |
| metP-metalloprotease | AGTCTCAACTCGCCGATCTG | AGACCTTCCAACTTGACGCC |
| | (SEQ ID NO: 22) | (SEQ ID NO: 23) |
| fruB-phosphocarrier | CTGGATGTGGCGACCGATAA | ACACCTTGCCCCAGATTCAG |
| protein | (SEQ ID NO: 24) | (SEQ ID NO: 25) |
| fliD-flagellar filament | CAACCAAAGTCACCAGCACG | TGCACTGGTTTTGGCTGTTG |
| capping protein | (SEQ ID NO: 26) | (SEQ ID NO: 27) |
| cheA-chemotaxis protein | TCTGCGGTGGGTAACTTGAC | TCACCGCTAAACCTTGGGAC |
| | (SEQ ID NO: 28) | (SEQ ID NO: 29) |
| artI-arginine ABC | GCACGTTTAGCAATCAGGCC | GTAAGGCTGGGTGAACGACA |
| transporter | (SEQ ID NO: 30) | (SEQ ID NO: 31) |

Primers used for cloning

| | | |
|---|---|---|
| g18987 | CGCCATATGATGTCGATTCCCCAAT | GGAGGATCCCTACGCCTTGATGAAC |
| | (SEQ ID NO: 32) | (SEQ ID NO: 33) |
| g28531 | CGCCATATGATGAAATCCATTAATATTTTGATG | GGAGGATCCCTAAGCATCCTTTGCCT |
| | (SEQ ID NO: 34) | (SEQ ID NO: 35) |
| g39249 | CGCCATATGATGGAGTCAAAGTTTGC | GGAGGATCCTTAAGCCACCCTAGGA |
| | (SEQ ID NO: 36) | (SEQ ID NO: 37) |
| g40384 | CGCCATATGATGAAGTCGATTAATATTTTGAG | GGAGGATCCCTACGCCTTGATGAACA |
| | (SEQ ID NO: 38) | (SEQ ID NO: 39) |
| g6571 | CGCCATATGATGAAGATCATAGTAGTACTC | GGAGGATCCTCAATAAGCTTGGGTCTT |
| | (SEQ ID NO: 40) | (SEQ ID NO: 41) |

### Proteomics Detection, Data Processing, and Annotation

The protein concentrations of each sample (DM1, M6, Pb1692+DM1/M6) were adjusted to 50 μg and processed by trypsin digestion. The samples were reconstituted in 8 M urea and 0.2% ProteaseMAX™ surfactant trypsin enhancer (Promega). Samples were further reduced and alkylated with 5 mM DTT and 5 mM iodoacetamide. Pierce MS-grade trypsin (Thermo Scientific) was added at an enzyme to substrate ratio of 1:50 and incubated at 37° C. for 3 h. Following incubation, trypsin was deactivated using 5% trifluoroacetic acid and desalted using Pierce C18 spin columns (Thermo Scientific) following the manufacturer's instructions. The eluted peptide samples were dried in a vacuum evaporator and resuspended in 5% acetonitrile/0.1% formic acid. Once resolubilized, absorbance at 250 nm was measured on a NanoDrop (Thermo Scientific) and the total peptide concentration was subsequently calculated using an extinction coefficient of 31.

Mass spectrometry analyses were performed using reverse phase liquid chromatography by using 0.1% formic acid (A) and acetonitrile with 0.1% formic acid (B). A total of 0.8 μg of peptides were purified and concentrated using an online enrichment column (Waters Symmetry Trap C18 100 Å, 5 μm, 180 μm ID×20 mm column). Subsequent chromatographic separation was performed on a reverse phase nanospray column (Waters, Peptide BEH C18; 1.7 μm, 75 μm ID×150 mm column, 45° C.) using a 90 min gradient: 5%-30% B over 85 min followed by 30%-45% B over 5 min (0.1% formic acid in ACN) at a flow rate of 350 nanoliters/min. Peptides were eluted directly into the mass spectrometer (Orbitrap Velos Pro, Thermo Scientific) equipped with a Nanospray Flex ion source (Thermo Scientific) and spectra were collected over a m/z range of 400-2000 under positive mode ionization. Ions with charge states +2 or +3 were accepted for MS/MS using a dynamic exclusion limit of 2 MS/MS spectra of a given m/z value for 30 s (exclusion duration of 90 s). The instrument was operated in FT mode for MS detection (resolution of 60,000) and ion trap mode for MS/MS detection with a normalized collision energy set to 35%. Compound lists of the resulting spectra were generated using Xcalibur 3.0 software (Thermo Scientific) with a signal to noise threshold of 1.5 and 1 scan/group.

Tandem mass spectra were extracted, charge state deconvoluted, and deisotoped by ProteoWizard MsConvert (version 3.0). Spectra from all samples were searched using Mascot (Matrix Science, London, UK; version 2.6.0) against reverse concatenated versions of the
CRAP_rev_100518, Uniprot_Solanum_Potato_rev_082819,
Custom_Solanum_chacoense_rev_082819,
Pbrasiliense1692_CodingProtein_CP047495_rev_012920, and
Uniprot_Pectobacterium_brasiliense_merge_rev_093019 databases (235,369 total entries)
assuming trypsin digestion. Mascot was searched with a fragment ion mass tolerance of 0.80 Da and a parent ion tolerance of 20 ppm. Carboxymethyl of cysteine was specified in Mascot as a fixed modification. Deamidation of asparagine and glutamine and oxidation of methionine were specified in Mascot as variable modifications.

Search results from all samples were imported and combined using the probabilistic protein identification algorithms implemented in the Scaffold software (version Scaffold_4.10.0, Proteome Software Inc., Portland, OR). Peptide thresholds were set at 95% so that a peptide false discovery rate (FDR) of 0.01% was achieved based on hits to the reverse database. Protein identifications were accepted if they could be established at greater than 95% probability and contained at least 2 identified peptides. Protein probabilities were assigned by the Protein Prophet algorithm. Also, the data was searched against a contaminant database and contaminants were removed. Proteins that contained similar peptides and could not be differentiated based on MS/MS analysis alone were grouped to satisfy the principles of parsimony. Quantitative values (obtained using normalized total spectra) were used for all downstream statistical analysis.

### Phylogenetic Analysis, Domain Annotation, and Genetic Mapping of Protease Inhibitors

Protease inhibitors detected from the proteomics analysis of potato were examined for domains using PI domains in Simple Modular Architecture Research Tool (SMART v8.0). Similarly, PIs from tomato have been characterized, and sequence data was included here to improve the analysis of potatoes. PI protein sequences from this potato study and tomato were analyzed using the ClustalW application of MEGA vX. The aligned sequences were subjected to a phylogenetic analysis using a maximum likelihood method and Jones-Taylor-Thornton (JTT) matrix-based model with 500 bootstrap replicates. Protein sequences were blasted against the whole M6 or DM1 genome sequence using CoGe Blast to identify their location in chromosomes. The location of all PIs was obtained from CoGe database (available on the World Wide Web at genomevolution.org/coge) and mapped using phenogram (available on the World Wide Web at visualization.ritchielab.org/phenograms/plot).

### PI Gene Cloning and Protein Purification

S. chacoense M6 protease inhibitors g18987, g28531, g39249, g40384, and g6571 were selected for cloning and protein purification. These PI genes were PCR-amplified from S. chacoense M6 cDNA. Primers were designed based on the M6 genome sequence and the expression plasmid pET16b (Table 2) using overhang sequences for BamH1 and NdeI endonucleases. The PCR products were purified, digested, and ligated to pET16b. E. coli BL21 (DE3) was transformed with pET16b::PI by heat shock (42° C. for 1 min) and transformants were selected on nutrient agar plates containing 100 μg·ml⁻¹ ampicillin. The cloned PI genes were verified by PCR and by sequencing of the insert for the presence of PI genes in frame with an N-terminus His-X10 present in the construct.

PIs were purified by growing the transformed E. coli BL21 cells (carrying pET16b::PI) in NB supplemented with ampicillin (100 μg·ml⁻¹) at 37° C. with aeration overnight. Two microliters of the overnight culture were added to 100 ml of fresh NB and shaken at 37° C. until optical density (OD₆₀₀) of 0.5 was achieved. The temperature was then lowered to 25° C. and 0.5 mM isopropyl-b-D-thiogalactopyranoside (IPTG) was added to induce PI production. After 5 h, and cells were harvested by centrifugation. Cell pellets were washed and resuspended in phosphate buffer saline (PBS) for protein purification as previously described. Briefly, cells were lysed by sonication (30 sec pulse of 25 kHz for 6 times with a 2 min resting period between each pulse). DNase was added to the sonicates and then clarified by centrifugation at 14,000 g for 20 min. The supernatants were loaded onto HisTrap columns (Thermo Fisher Scientific), and proteins were eluted following manufacturer's instructions. Eluted proteins were dialyzed in PBS using dialysis membrane.

Microscopy of P. brasiliense Pb1692

Bacterial cell morphology was observed under a compound microscope. Pb1692 cells were incubated with 400 μg·ml⁻¹ crude protein extract, cPI, cloned and purified M6 PI, or protein buffer as described above. Cells were pipetted to glass slides, heat fixed, stained with crystal violet, and observed under 1000× magnification.

### Virulence Assays

Virulence assays were conducted on potato tubers (S. tuberosum ‘Russet’). Tubers were washed and externally disinfected by spraying 70% ethanol and dried inside a Class II biosafety cabinet. A sterile cork-borer (5 mm diameter) was used to make 2 cm deep holes in tuber. A total of 10⁸ CFU of Pb1692 and 120 μg of cloned and purified PI protein was mixed with gentle pipetting and 300 μL of the mixture was pipetted into the hole. Potatoes were wrapped with clingfilm to prevent drying of the wound holes and incubated at 28° C. After 3 days, the potato tubers were sliced through the wound. Tubers were weighed, then macerated tissues were physically separated from the tuber with gentle scraping, and the remaining non-macerated tuber tissue was then weighed.

### Statistical Analysis

Bacterial growth rates, exo-enzyme, and gene expression data were analyzed using GraphPad Prism 8.0.1 (GraphPad Software Inc., CA, USA) using ANOVA with a Dunnett's post hoc test to compare individual treatments with the controls. ANOVA with a Tukey's post hoc or Student's t test were performed with JMP-Pro v5.0 (SAS Institute Inc., NC, USA) with a p threshold of 0.05. Protein normalized total spectra (NTS) abundances were compared between DM1 and M6 using Student's t tests, with a p threshold of 0.05, for all detected protein/peptide independently. Fold changes of detected proteins were calculated as log₂ of (mean M6/mean DM1). Presence of a protein was determined when at least two replicates had NTS value greater than zero. A residual value of 1 was applied to null NTS to calculate fold change. All graphs were illustrated using GraphPad Prism 8.0.1.

### Example 5: Effects of S. Chacoense PIs on a Broad Range of Pathogenic Bacteria

Proteases contribute to virulence for both gram-negative and gram-positive bacterial pathogens in plants and animals. Secreted Pectobacterium metalloproteases degrade plant cell wall proteins, such as extensins and lectins. In animal pathogens, similar proteases degrade host surfactant proteins, which are host defense proteins that can permeabilize bacterial membranes. Other cytoplasmic and periplasmic proteases in bacteria play a role in bacterial fitness since they are required to remove misfolded proteins and they contribute to bacterial gene regulation. Proteolysis is also important for flagellar secretion.

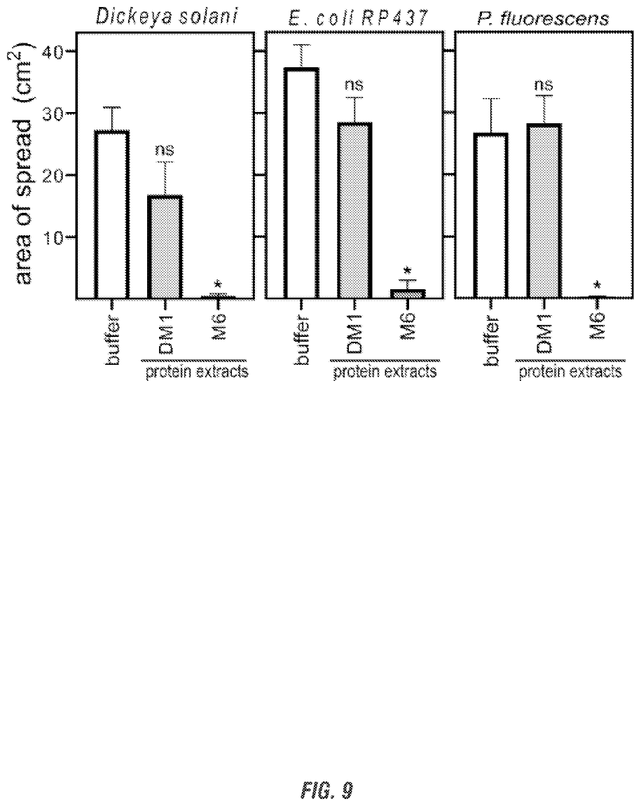

The S. chacoense protein extract was tested on additional bacterial species. Swimming and swarming motility assays showed effects on Dickeya solani, Eschericia coli, Pseudomonas syringae, and Pseudomonas fluorescens indicating avirulence activity of the protease inhibitors towards a broad range of pathogenic bacteria (FIG. 9).

1. A plant, or a plant cell thereof, with enhanced resistance to at least one bacterial pathogen, the plant comprising a heterologous polynucleotide encoding a protease inhibitor protein having at least 80%, at least 90%, at least 95%, at least 98%, or at least 99% sequence identity to at least one of the amino acid sequences set forth in SEQ ID NOs: 1-5.

2. The plant of claim 1, wherein the polynucleotide encoding the protease inhibitor protein has at least 80%, at least 90%, at least 95%, at least 98%, or at least 99% sequence identity to at least one of the nucleotide sequences set forth in SEQ ID NOs: 6-15.

3. The plant of claim 1, wherein the polynucleotide is operably linked to a promoter functional in a plant cell.

4. (canceled)

5. The plant of claim 1, wherein the at least one bacterial pathogen is of the family Pectobacteriaceae, or wherein the at least one bacterial pathogen causes a bacterial soft rot.

6.-8. (canceled)

9. The plant of claim 1, wherein the plant is a potato plant.

10. A fruit, tuber, leaf, or seed of the plant of claim 1, wherein the fruit, tuber, leaf, or seed comprises the heterologous polynucleotide.

11. A method of enhancing the resistance of a plant to at least one bacterial pathogen, the method comprising:
modifying at least one plant cell to comprise a polynucleotide encoding a protease inhibitor protein having at least 80%, at least 90%, at least 95%, at least 98%, or at least 99% sequence identity to at least one of the amino acid sequences set forth in SEQ ID NOs: 1-5.

12. (canceled)

13. The method of claim 11, further comprising regenerating a plant comprising in its genome the polynucleotide from the plant cell.

14. The method of claim 11, wherein modifying at least one plant cell to comprise the polynucleotide comprises (a) introducing a heterologous polynucleotide encoding the protease inhibitor protein into at least one plant cell, or (b) using genome editing to modify the nucleotide sequences of a native or non-native gene in the genome of the plant cell to comprise the polynucleotide encoding the protease inhibitor protein.

15.-18. (canceled)

19. The method of claim 11, further comprising selecting for a plant or a plant cell having enhanced resistance to at least one bacterial pathogen as compared to a corresponding control plant or plant cell without the polynucleotide.

20. The method of claim 11, wherein the at least one bacterial pathogen is of the family Pectobacteriaceae, or wherein the at least one bacterial pathogen causes a bacterial soft rot.

21.-23. (canceled)

24. The method of claim 11, wherein the plant is a potato plant.

25. An expression cassette comprising:
a nucleic acid molecule comprising a nucleotide sequence selected from the group of:
(a) the nucleotide sequence set forth in SEQ ID NO: 6, 7, 8, 9, 10, 11, 12, 13, 14, or 15;
(b) a nucleotide sequence encoding the amino acid sequence set forth in SEQ ID NO: 1, 2, 3, 4, or 5;
(c) a nucleotide sequence having at least 80%, at least 90%, at least 95%, at least 98%, or at least 99% sequence identity to at least one of the nucleotide sequences set forth in SEQ ID NOs: 6, 7, 8, 9, 10, 11, 12, 13, 14, and 15; and
(d) a nucleotide sequence encoding an amino acid sequence having at least 80%, at least 90%, at least 95%, at least 98%, or at least 99% sequence identity to at least one of the amino acid sequences set forth in SEQ ID NOs: 1, 2, 3, 4, and 5,
wherein the nucleic acid molecule is operably linked to a heterologous promoter.

26. (canceled)

27. A vector comprising the expression cassette of claim 25.

28. A host cell comprising the expression cassette of claim 25.

29.-31. (canceled)

32. A method of limiting a plant disease caused by at least one bacterial pathogen in agricultural crop production, the method comprising:
planting a seedling, tuber, or seed of the plant of claim 1; and
growing the seedling, tuber, or seed under conditions favorable for the growth and development of a plant resulting therefrom.

33. The method of claim 32, further comprising harvesting at least one fruit, tuber, leaf and/or seed from the plant.

34. A method for identifying a plant that displays newly conferred or enhanced resistance to a plant disease caused by at least one bacterial pathogen, the method comprising:
detecting in the plant, or in at least one part or cell thereof, the presence of a protease inhibitor nucleotide sequence having at least 80%, at least 90%, at least 95%, at least 98%, or at least 99% sequence identity to at least one of the nucleotide sequences set forth in SEQ ID NOs: 6-15.

35. The method of claim 34, wherein the plant disease is a bacterial soft rot or blackleg.

36. (canceled)

37. The method of claim 34, wherein the plant is a potato plant.

38.-39. (canceled)

40. A method for introducing at least one protease inhibitor gene into a plant, the method comprising:
(a) crossing a first plant comprising in its genome at least one copy of at least one protease inhibitor gene encoding a protease inhibitor protein having at least 80%, at least 90%, at least 95%, at least 98%, or at least 99% sequence identity to at least one of the amino acid sequences set forth in SEQ ID NOs: 1-5 with a second plant lacking in its genome the at least one protease inhibitor gene, whereby at least one progeny plant is produced; and
(b) selecting at least one progeny plant comprising in its genome the at least one protease inhibitor gene.

41. The method of claim 40, wherein the first plant is a Solanum chocoense plant and the second plant is not a Solanum chocoense plant, or wherein the second plant is a Solanum tuberosum plant.

42.-43. (canceled)

44. The method of claim 38, further comprising (i) backcrossing at least one selected progeny plant of (b) to a plant that is of the same species and genotype as second plant or of the same species as the second plant and lacking in its genome the at least one protease inhibitor gene, whereby at least one progeny plant is produced from the backcrossing; and (ii) selecting at least one progeny plant comprising in its genome the at least one protease inhibitor gene that is produced from the backcrossing of (i).

45. An antimicrobial composition comprising:
at least one protease inhibitor protein having at least 80%, at least 90%, at least 95%, at least 98%, or at least 99% sequence identity to at least one of the amino acid sequences set forth in SEQ ID NOs: 1-5.

46. The antimicrobial composition of claim 45, wherein the composition comprises two, three, four, or five protease inhibitor proteins having an amino acid sequence selected from SEQ ID NOs: 1-5.

47. (canceled)

48. The antimicrobial composition of claim 45, further comprising one or more of a carrier, a filler, a diluent, a dye, an adjuvant, an emulsifier, a dispersing agent, a wetting agent, a thickener, a thixotropic agent, or a defoaming agent.

49. (canceled)

50. A method of preventing or controlling microbial growth on a plant or a plant part, the method comprising:
contacting the surface of the plant or plant part with the antimicrobial composition of claim 45.

51. The method of claim 50, wherein the plant or plant part is dipped in the antimicrobial composition, or wherein the plant or plant part is sprayed or coated with the antimicrobial composition.

52. (canceled)

53. The method of claim 50, wherein the plant part is a harvested plant part, or wherein the plant part is a fruit, tuber, leaf, or seed.

54. (canceled)

55. The method of claim 50, wherein the plant or plant part is a potato plant or potato plant part.
